SEQUENCE LISTING (1 sequence)

SEQ ID NO: 1 - protein, 415 aa, Homo Sapiens
YNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAEAVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLT

(19) United States
(12) Patent Application Publication
(10) Pub. No.: US 2017/0035890 A1
(43) Pub. Date: Feb. 9, 2017

(54) Coagulation Factor IX Conjugates

(71) Applicant: NOVO NORDISK HEALTH CARE AG, Zürich (CH)

(72) Inventors: Carsten Behrens, Koebenhavn (DK); Paul Deangelis, Edmond, OK (US); Friedrich Michael Haller, Norman, OK (US)

(21) Appl. No.: 15/117,922

(22) PCT Filed: Feb. 12, 2015
(86) PCT No.: PCT/EP2015/053030

§ 371 Date: Aug. 10, 2016

(30) Foreign Application Priority Data
Feb. 12, 2014 (EP) 14154874.3

(51) Int. Cl. A61K 47/48; C12N 9/64; C12N 9/96; A61K 38/48

(52) U.S. Cl. CPC A61K 47/4823; A61K 38/4846; C12Y 304/21022; C12N 9/644; C12N 9/96

(57) ABSTRACT

The present invention relates to Factor IX polypeptides conjugated to heparosan (HEP) polymers, methods for the manufacture thereof and uses of such conjugates. The resultant conjugates may be used—for example—in the treatment or prevention of bleeding disorders such as haemophilia B.

### FIELD OF THE INVENTION

The present invention relates to conjugates between blood coagulation Factor IX and heparosan polymers and uses thereof.

### BACKGROUND

In subjects with a coagulopathy, such as in human beings with haemophilia, various steps of the coagulation cascade are rendered dysfunctional due to, for example, the absence or insufficient presence of a coagulation factor. Such dysfunction of one part of the coagulation cascade results in insufficient blood coagulation and potentially life-threatening bleeding, or damage to internal organs, such as the joints.

Haemophilia B is caused by deficiency or dysfunction of coagulation Factor IX activity and patients can be treated by on demand administration of Factor IX.

Current treatment recommendations are moving from traditional on-demand treatment towards prophylaxis. Current prophylaxis therapy requires multiple dosing a week, but for optimal plasma levels and efficacy, once-daily injections would be superior. Due to the practical and economical limitations associated with daily administrations, this is not an ideal solution for the patients.

Coagulation Factor IX is a valuable therapeutic polypeptide for use in the treatment of haemophilia B. Although commercially available forms of Factor IX are in use today there remains a general need in the art for longer lasting Factor IX polypeptides with improved pharmacokinetics.

Therapeutic polypeptides, such as Factor IX polypeptides can be fused or conjugated to half-life extending moieties in order to extend the plasma half-life of said medicament after being administrated to a patient.

Conjugation of half-life extending moieties, e.g. in the form of a hydrophilic polymer, with a peptide or polypeptide can be carried out by use of enzymatic methods. These methods can be selective, requiring the presence of specific peptide consensus motives in the protein sequence, or the presence of post translational moieties such as glycans.

Selective enzymatic methods for modifying N- and O-glycans on blood coagulation factors have been described. For example, chemically modified sialic acid substrates (Malmstrøm, J Anal Bioanal Chem 2012; 403:1167-1177) have been described that can be used to glycoPEGylate Factor VIIa on N-glycans using sialyltransferase ST3GaIII (Stennicke, H R. et al. Thromb Haemost. 2008 November; 100(5):920-8), and on O-glycans on Factor VIII using ST3GaII (Stennicke, H R. et al., Blood. 2013 Mar. 14; 121(11):2108-16).

US2006040856, for example, relates to conjugates between Factor IX and polyethylene glycol (PEG) moieties linked via an intact glycosyl linking group interposed between and covalently attached to the peptide and the PEG moiety.

A common feature of the above mentioned methods is the use of a modified sialic acid substrate, glycyl sialic acid cytidine monophosphate (GSC), and the chemical acylation of GSC with the half-life extending moieties.

For example, PEG polymers activated as nitrophenyl- or N-hydroxy-succinimide esters can be acylated onto the glycyl amino group of GSC to create a PEG substituted sialic acid substrate that can be enzymatically transferred to the N- and O-glycans of glycoproteins (cf. WO2006127896, WO2007022512, US2006040856). In a similar way, fatty acids can be acylated onto the glycyl amino group of GSC using N-hydroxy-succinimide activated ester chemistry (WO2011101277).

However, the inventors have found that the previously published methods are not suited for attaching highly functionalized half-life extending moieties such as carbohydrate polymers to GSC.

In the present invention, novel conjugates between the half-life extending polymer heparosan and Factor IX as well as uses and methods for the production thereof, are disclosed.

### SUMMARY OF THE INVENTION

Described herein are novel heparosan-Factor IX (HEP-FIX) polypeptide conjugates and preparations thereof. These conjugates provide biological properties superior to certain other conjugates known in the art e.g. PEG-based conjugates.

The conjugates described herein are protected by a biodegradable half-life extending moiety in the form of heparosan (HEP) which extends the in vivo half-life of Factor IX (FIX). In some embodiments the HEP-FIX polypeptide conjugate of the invention has increased circulation half-life compared to an un-conjugated FIX polypeptide; or increased functional half-life compared to an un-conjugated FIX polypeptide.

In some embodiments the HEP-FIX polypeptide conjugate has increased mean residence time compared to an un-conjugated FIX polypeptide; or increased functional mean residence time compared to an un-conjugated FIX polypeptide.

Moreover, in some embodiments the conjugates show improved performance inter alia compared to similar PEGylated FIX variants in aPTT assays.

In some embodiments HEP in the form of a polymer has a polydispersity index (Mw/Mn) of less than 1.10 or less than 1.05.

In one embodiment, the polymer may have an average size between approximately 13 and approximately 60 kDa, such as 38, 41 and 44 kDa.

Also, the HEP-FIX polypeptide conjugates described herein can be produced using a linker which has improved properties (e.g., stability). In one such embodiment HEP-FIX polypeptide conjugates are provided wherein the HEP moiety is linked to FIX in such a fashion that a stable and isomer free conjugate is obtained. In one such embodiment the HEP polymer is linked to FIX using a chemical linker comprising 4-methylbenzoyl connected to a sialic acid derivative such as glycyl sialic acid cytidine monophosphate (GSC).

The HEP-FIX polypeptide conjugates described herein are particularly useful in the treatment of coagulopathy and in particular prophylactic treatment of haemophilia B.

### BRIEF DESCRIPTION OF DRAWINGS

FIG. 1: Functionalization of glycyl sialic acid cytidine monophosphate (GSC) with a benzaldehyde group. GSC is acylated with 4-formylbenzoic acid and subsequently reacted with heparosan (HEP)-amine by a reductive amination reaction.

FIG. 2: Functionalization of heparosan (HEP) polymer with a benzaldehyde group and subsequent reaction with glycyl sialic acid cytidine monophosphate (GSC) in a reductive amination reaction.

FIG. 3: Functionalization of glycyl sialic acid cytidine monophosphate (GSC) with a thiol group and subsequent reaction with a maleimide functionalized heparosan (HEP) polymer.

FIG. 4: Heparosan (HEP)-glycyl sialic acid cytidine monophosphate (GSC).

FIG. 5: Heparosan (HEP)-glycyl sialic acid cytidine monophosphate (GSC) conjugated onto a biantenna N-glycan on Factor IX (pos. N157 or N167) via a 4-methylbenzoyl sublinkage.

FIG. 6: Plasma FIX concentrations versus time in F9-KO mice. The concentrations were measured by an antigen based assay (a) as well as clot activity and chromogenic activity based assays (b) and (c), respectively) versus time. F9-KO mice were dosed IV with 27 nmol/kg (1.5 mg FIX/kg) of BeneFIX®, 40 kDa PEG-[N]-FIX and 60 kDa HEP-[C]-FIX(E162C). Results are mean±SD in a semi-logarithmic plot, n=3.

FIG. 7: Plasma concentrations of FIX versus time in F9-KO mice. The concentrations were measured by an antigen based assay (FIG. 7a) and chromogenic activity based assays (FIG. 7b). The mice were dosed IV with 27 nmol/kg (1.5 mg FIX/kg) of FIX conjugated to 13 to 60 kDa HEP polymers. “C” indicates Cys-conjugation and “N” indicates N-glycan conjugation. Results are mean±SD in a semi-logarithmic plot, n=3.

FIG. 8: 60 kDa HEP-[C]-FIX E162C and FIX dose-dependently and significantly reduced blood loss after tail vein transection in F9-KO mice with comparable potency. The F9-KO mice were dosed 10 min before induction of bleeding. ED₅₀ was 0.012 mg/kg and 0.030 mg/kg for FIX-HEP and FIX, respectively (p=0.38). * and *** indicate statistical significant difference at p<0.05 and 0.001, respectively, compared to the haemophilia control group receiving vehicle. Data are mean±SEM.

FIG. 9: 60 kDa HEP-[C]-FIX E162C (FIX-HEP) and rFIX dose-dependently and significantly reduced bleeding time after tail vein transection in F9-KO mice with comparable potency. The F9-KO mice were dosed 10 min before induction of bleeding. ED₅₀ was 0.009 mg/kg and 0.024 mg/kg for FIX-HEP and FIX, respectively (p=0.18). *, ** and *** indicate statistical significant difference at p<0.05, 0.01 and 0.001, respectively, compared to the haemophilia control receiving vehicle. Data are mean±SEM.

FIG. 10 40 kDa HEP-[N]-FIX and rFIX dose-dependently and significantly reduced the blood loss after tail vein transection in F9-KO mice with comparable potency. The F9-KO mice were dosed 10 min before induction of bleeding. ED₅₀ was 0.032 mg/kg and 0.027 mg/kg for kDa HEP-[N]-FIX and rFIX, respectively (p=0.67). *** and **** indicate statistical significant difference at p<0.001 and 0.0001, respectively, compared to the haemophilia control receiving vehicle. Data are mean±SEM

FIG. 11: Recovery of FIX activity in spiked human FIX deficient plasma relative to chromogenic activity. Three concentrations of compounds were spiked into human FIX depleted plasma and analysed using the Biophen Hypen chromogenic assay and five specified aPTT reagents in the one-stage clot assay. Results are given as clot activity in percent of chromogenic activity and are mean+/−SD, n=3. Activity was measured against a normal human plasma calibrator (ILS) in all assays.

Compound: columns 1-5: BeneFIX®; columns 6-10: 27 kDa HEP-[C]-FIX(E162C); columns 11-15: 40 kDa HEP-[C]-FIX(E162C); columns 16-20: 40 kDa HEP-[N]-FIX; columns 21-25: 60 kDa HEP-[C]-FIX(E162C); columns 26-30: N9-GP).

Type of aPTT-based assay: columns 1, 6, 11, 16, 21, 26: Actin FS® (Siemens); columns 2, 7, 12, 17, 22, 27: Synthasil® (ILS); columns 3, 8, 13, 18, 23, 28: Synthafax® (ILS); columns 4, 9, 14, 19, 24, 29: APTT SP (ILS); columns 5, 10, 15, 20, 25, 30: STA PTT® (Stago).

FIG. 12: This figure shows the significantly extended duration of the haemostatic effect of 40 kDa HEP-[N]-FIX compared to an equivalent dose of rFIX. Both compounds significantly reduced the blood loss caused by tail vein transection in F9-KO mice immediately after dosing, but 72 hours after dosing the effect of 40 kDa HEP-[N]-FIX was still significant compared to the vehicle group (P=0.0028) and the rFIX treated group (P=0.022). *, ** and **** indicate statistical significant difference at p<0.05, 0.01 and 0.0001, respectively. Data are mean±SEM.

FIG. 13: Reaction scheme wherein an asialoFIX glycoprotein is reacted with HEP-GSC in the presence of a ST3GaIII sialyltransferase.

### BRIEF DESCRIPTION OF THE SEQUENCE

SEQ ID NO: 1 gives the amino acid sequence of human Factor IX.

### DESCRIPTION

The present invention is directed to novel heparosan—Factor IX polypeptide (HEP-FIX) conjugates and preparations thereof. These conjugates provide biological properties superior to other conjugates known in the art.

Factor IX (FIX) deficiency, commonly referred to as haemophilia B, is a congenital bleeding disorder affecting approximately 120,000 people worldwide, of which around 27,000 are currently diagnosed and less than 10,000 receive care. Conventional treatment consists of replacement therapy, provided as prophylaxis or on demand treatment of bleeding episodes. The current treatment for a person with severe haemophilia B is usually 2-3 prophylactic injections/week of BeneFIX® (wild type rFIX).

In subjects with a coagulopathy, such as in human beings with haemophilia A and B, various steps of the coagulation cascade are rendered dysfunctional due to, for example, the absence or insufficient presence of a coagulation factor. Such dysfunction of one part of the coagulation cascade results in insufficient blood coagulation and potentially life-threatening bleeding, or damage to internal organs, such as the joints. Individuals with haemophilia B may receive coagulation factor replacement therapy such as exogenous FIX. However, such patients are at risk of developing neutralizing antibodies, so-called “inhibitors”, to such exogenous factors, rendering formerly efficient therapy ineffective. Furthermore, exogenous coagulation factors may only be administered intravenously, which is of considerable inconvenience and discomfort to patients. For example, infants and toddlers may have to have intravenous catheters surgically inserted into a chest vein, in order for venous access to be guaranteed. This leaves them at great risk of developing bacterial infections. There are thus still many unmet medical needs in the haemophilia community, in particular, and in subjects with coagulopathies, in general.

Congenital hypocoagulopathies include haemophilia B. Said haemophilia B may be severe, moderate or mild. The clinical severity of haemophilia is determined by the concentration of functional units of FIX in the blood and is classified as mild, moderate, or severe. Severe haemophilia is defined by a clotting factor level of <0.01 U/ml corresponding to <1% of the normal level, while moderate and mild patients have levels from 1-5% and >5%, respectively.

Congenital deficiency of FIX activity is the cause of the X-linked bleeding disorder haemophilia B affecting approximately 1 in 100000 males. These haemophilia B patients are currently treated by replacement therapy with either recombinant or plasma-derived Factor IX.

Haemophilia B with “inhibitors” (that is, allo-antibodies against FIX) is a non-limiting example of a coagulopathy that is partly congenital and partly acquired.

A non-limiting example of an acquired coagulopathy is serine protease deficiency caused by vitamin K deficiency; such vitamin K-deficiency may be caused by administration of a vitamin K antagonist, such as warfarin. Acquired coagulopathy may also occur following extensive trauma. In this case otherwise known as the “bloody vicious cycle”, it is characterised by haemodilution (dilutional thrombocytopaenia and dilution of clotting factors), hypothermia, consumption of clotting factors and metabolic derangements (acidosis). Fluid therapy and increased fibrinolysis may exacerbate this situation. Said haemorrhage may be from any part of the body.

A non-limiting example of an iatrogenic coagulopathy is an overdosage of anticoagulant medication—such as heparin, aspirin, warfarin and other platelet aggregation inhibitors—that may be prescribed to treat thromboembolic disease. A second, non-limiting example of iatrogenic coagulopathy is that which is induced by excessive and/or inappropriate fluid therapy, such as that which may be induced by a blood transfusion.

In one embodiment of the current invention, haemorrhage is associated with haemophilia B. In another embodiment, haemorrhage is associated with haemophilia B with acquired inhibitors. In another embodiment, haemorrhage is associated with thrombocytopenia. In another embodiment, haemorrhage is associated with von Willebrand's disease. In another embodiment, haemorrhage is associated with severe tissue damage. In another embodiment, haemorrhage is associated with severe trauma. In another embodiment, haemorrhage is associated with surgery. In another embodiment, haemorrhage is associated with haemorrhagic gastritis and/or enteritis. In another embodiment, the haemorrhage is profuse uterine bleeding, such as in placental abruption. In another embodiment, haemorrhage occurs in organs with a limited possibility for mechanical haemostasis, such as intracranially, intraaurally or intraocularly. In another embodiment, haemorrhage is associated with anticoagulant therapy.

### Factor IX

FIX is a vitamin K-dependent coagulation factor with structural similarities to Factor VII, prothrombin, Factor X, and Protein C. The circulating zymogen form consists of 415 amino acids divided into four distinct domains comprising an N-terminal γ-carboxyglutamic acid-rich (Gla) domain, two EGF domains and a C-terminal trypsin-like serine protease domain. One example of a “wild type FIX” is the full length human FIX molecule, as shown in SEQ ID NO: 1.

Activation of FIX occurs by limited proteolysis at Arg¹⁴⁵-Ala¹⁴⁶ and Arg¹⁸⁰-Val¹⁸¹ releasing a 35-aa fragment, the so-called activation peptide. The activation peptide is heavily glycosylated, containing two N-linked (in positions N157 and N167) and several O-linked glycans. Activated Factor IX is referred to as Factor IX(a) or FIX(a). FIX(a) is a trypsin-like serine protease that serves a key role in haemostasis by generating, as part of the tenase complex, most of the Factor Xa required to support proper thrombin formation during coagulation. “FIX(a)” includes natural allelic variants of FIX(a) that may exist and occur from one individual to another.

Unless otherwise specified, FIX domains include the following amino acid residues: Gla domain being the region from reside Tyr1 to residue Lys43; EGF1 being the region from residue Gln44 to residue Leu84; EGF2 being the region from residue Asp85 to residue Arg145; the Activation Peptide being the region from residue Ala146 to residue Arg180; and the Protease Domain being the region from residue Val181 to Thr414. The light chain refers to the region encompassing the Gla domain, EGF1 and EGF2, while the heavy chain refers to the Protease Domain. The sequence of wild type human coagulation FIX is listed below:

YNSGKLyyFVQGNLyRyCMyyKCSFyyARyVFyNTyRTTyFWKQYVDGDQ
CESNPCLNGGSCKDDINSYECWCPFGFEGKNCELDVTCNIKNGRCEQFCK
NSADNKVVCSCTEGYRLAENQKSCEPAVPFPCGRVSVSQTSKLTRAEAVF
PDVDYVNSTEAETILDNITQSTQSFNDFTRVVGGEDAKPGQFPWQVVLNG
KVDAFCGGSIVNEKWIVTAAHCVETGVKITVVAGEHNIEETEHTEQKRNV
IRIIPHHNYNAAINKYNHDIALLELDEPLVLNSYVTPICIADKEYTNIFL
KFGSGYVSGWGRVFHKGRSALVLQYLRVPLVDRATCLRSTKFTIYNNMFC
AGFHEGGRDSCQGDSGGPHVTEVEGTSFLTGIISWGEECAMKGKYGIYTK
VSRYVNWIKEKTKLT

wherein γ represents gamma-carboxylated Glu (‘E’). In fully gamma-carboxylated FIX, the first 12 Glu residues are gamma-carboxylated, but there are variants (especially in the case of recombinant FIX) in which less gamma-carboxylation takes place. A dimorphism is present in FIX at position 148, which can be either Ala or Thr (see McGraw et al. (1985) PNAS, 82:2847).

“Factor IX” or “FIX”, as used herein, refer to a human plasma FIX glycoprotein that is a member of the coagulation contact activation pathway (also known as the intrinsic coagulation pathway) and is essential to blood coagulation. Unless otherwise specified or indicated, FIX means any functional human FIX protein molecule in its normal role in coagulation.

The term “FIX analogue”, as used herein, is intended to designate FIX having the sequence of SEQ ID NO: 1, except that one or more amino acids of FIX have been substituted by another amino acid and/or wherein one or more amino acids of FIX have been deleted and/or wherein one or more amino acids have been inserted in FIX and/or wherein one or more amino acids have been added to FIX. Such addition can take place either at the N-terminal end or at the C-terminal end of the parent protein or both. The “analogue” or “analogues” within this definition still have FIX activity in its activated form. In one embodiment a variant is at least 90% identical with the sequence of SEQ ID NO: 1. In a further embodiment a variant is at least 95% identical with the sequence of SEQ ID NO: 1. As used herein any reference to a specific positions refers to the corresponding position in SEQ ID NO: 1.

As used herein, the terms “Factor IX polypeptide” or “FIX polypeptide” encompass, without limitation, wild-type human FIX and FIX(a) as well as polypeptides exhibiting substantially the same or improved biological activity relative to wild-type human FIX. These polypeptides include, without limitation, FIX or FIXa that has been chemically modified and FIX or FIXa analogues into which specific amino acid sequence alterations have been introduced that modify the bioactivity of the polypeptide unless otherwise indicated. Also encompassed are polypeptides with a modified amino acid sequence, for instance, polypeptides having a modified N-terminal end including N-terminal amino acid deletions or additions relative to human FIX. Also encompassed are polypeptides with a modified amino acid sequence, for instance, polypeptides having a modified C-terminal end including C-terminal amino acid deletions or additions, relative to human FIX.

FIX polypeptides, including analogues, variants and derivatives of FIX, exhibiting substantially the same or better bioactivity than wild-type FIX, include, without limitation, polypeptides having an amino acid sequence that differs from the sequence of wild-type FIX by addition, insertion, deletion, or substitution of one or more amino acids.

The present invention is in no way limited to the sequence set forth herein. FIX analogues are disclosed for example in U.S. Pat. No. 5,521,070 in which a tyrosine is replaced by an alanine in the first position and in WO2007/135182 in which one or more of the natural amino acid residues in FIX are substituted with a cysteine residue. Said references are hereby incorporated by reference in their entirety. Hence, analogues and variants of FIX are well known in the art, and the present disclosure encompasses those analogues or variants known or to be developed or discovered in the future.

FIX or FIX(a) may be plasma-derived or recombinantly produced using well known methods of production and purification. The degree and location of glycosylation, gamma-carboxylation and other post-translation modifications may vary depending on the chosen host cell and its growth conditions.

Host cells for producing recombinant proteins are preferably of mammalian origin in order to ensure that the molecule is properly processed during folding and post-translational modification, e.g. O and N-glycosylation and sulfatation. Suitable host cells include, without limitation, Chinese Hamster Ovary (CHO), baby hamster kidney (BHK), and HEK293 cell lines.

In some embodiments, pharmaceutical compositions, for example in the form of a formulation comprising FIX polypeptides conjugated to HEP are used to treat a subject with a coagulopathy, said coagulopathy for example being haemophilia B.

In some embodiments, compositions and formulations comprising HEP-FIX conjugates are provided. Specific embodiments include a pharmaceutical composition that comprises a HEP-FIX conjugates described herein, formulated together with a pharmaceutically acceptable carrier.

A major inhibition to the therapeutic use of clotting factors such as Factor IX is cost, particularly due to the effective dose of these proteins is high. A common dosage is 250 μg of protein/kg body weight.

One solution to the problem of providing cost effective glycopeptide therapeutics has been to provide peptides with longer in vivo half-lives. For example, glycopeptide therapeutics with improved pharmacokinetic properties have been produced by attaching synthetic polymers to the peptide backbone. An exemplary polymer that has been conjugated to peptides is poly(ethylene glycol) (PEG).

Present treatment of haemophilia B with FIX normally includes around two weekly injections supplemented with injections on an as-needed basis, e.g. before tooth extractions or surgery. FIX circulates as an inactive zymogen and is only converted in the active form Factor IX(a) when a bleed is to be arrested. Thus one way to accomplish a prophylactic FIX treatment based on e.g. one weekly injection is to increase the circulation time of FIX in the blood stream of the patient. In this way there will always be a certain level of zymogen FIX ready to be activated to ensure normal blood clotting conditions in the patient at any time.

Half-life extending moieties, alternatively referred to as side chains or side groups, may include biocompatible fatty acids and derivatives thereof and hydrophilic polymers such as Hydroxy Ethyl Starch, PEG, hyaluronic acid and HEP polymers. PEGylation has for years been one of the preferred half-life extension technologies for generating long acting drugs, and several PEG-protein conjugates have now reached the market. PEG polymers have a tendency to lower the activity of the protein drug to which it is bound. This typically results in lower drug-receptor affinity or lower binding affinity to the respective drug binding partners in solution. In most cases, the lowering of activity correlate with either PEG size or number of PEG groups attached to the protein drug. Thus attachment of large PEG groups leads to considerable higher activity loss than attachment of small PEG groups.

Beside the activity modulating effect of PEG size and PEG numbers, PEG has recently been shown to have strong interference with standard assays used in haemostasis. For example the specific activity of glycoPEGylated FVIII measured in one-stage clotting assays vary depending on the aPTT reagent used (Stennicke, Blood 2013; 121(11):2108-16).

Use of the aPTT one-stage FIX clotting assay is the standard procedure used for individual optimisation of the dose- and dosing regimens during initiation of treatment and for routine monitoring of FIX prophylaxis. In general, aPTT assays are conducted at a central laboratory where clotting of blood obtained from the patient is initiated by addition of an aPTT reagent and re-calcification after which time to fibrin clot formation is measured on a coagulation analyser. There are many commercially available formats of this assay.

The assay interfering property of PEG may have significant impact in preclinical development and even more so in clinical application where precise measurement of patients' blood coagulation factors in multi component one-stage clotting assay are required.

### Heparosan

Heparosan (HEP) is a natural sugar polymer comprising (-GlcUA-1,4-GlcNAc-1,4-) repeats. It belongs to the glycosaminoglycan polysaccharide family and is a negatively charged polymer at physiological pH. HEP can be found in the capsule of certain bacteria but it is also found in higher vertebrate where it serves as precursor for the natural polymers heparin and heparan sulphate. HEP can be degraded by lysosomal enzymes such as N-acetyl-a-D-glucosaminidase (NAGLU) and β-glucuronidase (GUSB). Some embodiments provide a heparosan polymer of the formula (-GlcUA-beta1,4-GlcNAc-alpha1,4-)ₙ. The size of the HEP polymer may be defined by the number of repeats n. The number of said repeats n may be, for example, from 2 to about 5,000. The number of repeats may be, for example 50 to 2,000 units, such as 105 units, 100 to 1,000 units, 5 to 450 or 200 to 700 units. The number of repeats may be 200 to 250 units, 500 to 550 units or 350 to 400 units. Any of the lower limits of these ranges may be combined with any higher upper limit of these ranges to form a suitable range of numbers of units in the HEP polymer.

The size of the HEP polymer may also be defined by its molecular weight. The molecular weight may be the average molecular weight for a population of HEP polymer molecules, such as the weight average molecular mass.

Molecular weight values as described herein in relation to size of the HEP polymer may not, in practise, exactly be the size listed. Due to batch to batch variation during HEP polymer production, some variation is to be expected. To encompass batch to batch variation, it is therefore to be understood, that a variation around +/−10%, 9%, 8%, 7%, 6%, 5%, 4%, 3%, 2% or 1% around target HEP polymer size is to be expected. For example HEP polymer size of 40 kDa denotes 40 kDa+/−10%, e.g. 40 kDa could for example in reality mean 38.8 kDa or 41.5 kDa, both falling within a +/−10% range of 36 to 44 kDa of 40 kDa.

In some embodiments the HEP polymer has a molecular weight of 500 Da to 1,000 kDa. In other embodiments the molecular weight of the polymer is 500 Da to 650 kDa, 5 to 750 kDa, 10 to 500 kDa, 15 to 550 kDa, 25 to 250 kDa or 50 to 175 kDa.

In some embodiments the molecular weight is selected at particular levels within the foregoing ranges in order to achieve a suitable balance between activity of the FIX polypeptide and half-life of the conjugate. For example, the molecular weight of the HEP polymer may be in a range selected from 5 to 15 kDa, 15 to 25 kDa, 25 to 35 kDa, 35 to 45 kDa, 45 to 55 kDa, 55 to 65 kDa, 65 to 75 kDa, 75 to 85 kDa, 85 to 95 kDa, 95 to 105 kDa, 105 to 115 kDa, 115 to 125 kDa, 125 to 135 kDa, 135 to 145 kDa, 145 to 155 kDa, 155 to 165 kDa or 165 to 175 kDa. In other embodiments, the molecular weight may be 500 Da to 21 kDa, such as 1 kDa to 15 kDa, such as 5 to 15 kDa, such as 8 to 17 kDa, such as 10 to 14 kDa such as about 12 kDa. The molecular weight may be 20 to 35 kDa, such as 22 to 32 kDa such as 25 to 30 kDa, such as about 27 kDa. The molecular weight may be 35 to 65 kDa, such as 40 to 60 kDa, such as 47 to 57 kDa, such as 50 to 55 kDa such as about 52 kDa. The molecular weight may be 50 to 75 kDa such as 60 to 70 kDa, such as 63 to 67 kDa such as about 65 kDa. The molecular weight may be 75 to 125 kDa, such as 90 to 120 kDa, such as 95 to 115 kDa, such as 100 to 112 kDa, such as 106 to 110 kDa such as about 108 kDa. The molecular weight may be 125 to 175 kDa, such as 140 to 165 kDa, such as 150 to 165 kDa, such as 155 to 160 kDa such as about 157 kDa. The molecular weight may be 5 to 100 kDa, such as 13 to 60 kDa and such as 27 to 40 kDa.

In some embodiments, the HEP polymer conjugated to the FIX polypeptide has a size in a range selected from 13 to 65 kDa, 13 to 60 kDa, 13 to 55 kDa, 13 to 50 kDa, 13 to 49 kDa, 13 to 48 kDa, 13 to 47 kDa, 13 to 46 kDa, 13 to 45 kDa, 13 to 44 kDa, 13 to 43 kDa, 13 to 42 kDa, 13 to 41 kDa, 13 to 40 kDa, 13 to 39 kDa, 13 to 38 kDa, 13 to 37 kDa, 13 to 36 kDa, 13 to 35 kDa, 13 to 34 kDa, 13 to 33 kDa, 13 to 32 kDa, 13 to 31 kDa, 13 to 30 kDa, 13 to 29 kDa, 13 to 28 kDa, 13 to 27 kDa, 13 to 26 kDa, 13 to 25 kDa, 13 to 21 kDa, 25 to 55 kDa, 25 to 50 kDa, 25 to 45 kDa, 27 to 40 kDa, 27 to 41 kDa, 27 to 42 kDa, 27 to 43 kDa, 27 to 43 kDa, 27 to 44 kDa, 27 to 45 kDa, 27 to 60 kDa, 30 to 45 kDa, 36 to 44 kDa and 38 to 42 kDa.

Any of the lower limits of these ranges of molecular weight may be combined with any higher upper limit from these ranges to form a suitable range for the molecular weight of the HEP polymer as described herein.

In connection with FIX polypeptide conjugates as described herein, use of HEP in the side chain offers a very flexible way of prolonging in vivo circulation half-life since a ranges of HEP sizes result in a significantly improved half-life.

HEP-polymers become highly viscous at high mass concentrations.

The HEP polymer may have a narrow size distribution (i.e. monodisperse) or a broad size distribution (i.e. polydisperse). The level of polydispersity may be represented numerically based on the formula Mw/Mn, where Mw=weight average molecular mass and Mn=number average molecular weight. The polydispersity value using this equation for an ideal monodisperse polymer is 1. Preferably, a HEP polymer is monodisperse. The polymer may therefore have a polydispersity that is about 1, the polydispersity may be less than 1.25, preferably less than 1.20, preferably less than 1.15, preferably less than 1.10, preferably less than 1.09, preferably less than 1.08, preferably less than 1.07, preferably less than 1.06, preferably less than 1.05. The molecular weight size distribution of the HEP may be measured by comparison with monodisperse size standards (HA Lo-Ladder, Hyalose LLC) which may be run on agarose gels.

Alternatively, the size distribution of HEP polymers may be determined by high performance size exclusion chromatography-multi angle laser light scattering (SEC-MALLS).

Such a method can be used to assess the molecular weight and polydispersity of a HEP polymer. Polymer size may be regulated in enzymatic methods of production. By controlling the molar ratio of HEP acceptor chains to UDP sugar, it is possible to select a final HEP polymer size that is desired.

HEP polymers can be prepared by a synchronised enzymatic polymerisation reaction (US20100036001). This method use heparan synthetase I from Pasteurella multocida (PmHS1) which can be expressed in E. coli as a maltose binding protein fusion constructs. Purified MBP-PmHS1 is able to produce monodisperse polymers in a synchronized, stoichiometrically controlled reaction, when it is added to an equimolar mixture of sugar nucleotides (GlcNAc-UDP and GlcUA-UDP). A trisaccharide initiator (GlcUA-GlcNAc-GlcUA) may be used to prime the reaction, and polymer length is determined by the primer:sugar nucleotide ratios. The polymerization reaction typically run until about 90% of the sugar nucleotides are consumed. Polymers are isolated from the reaction mixture by anion exchange chromatography, and subsequently freeze-dried into a stable powder.

### Methods for Preparing HEP-FIX Conjugates

In some embodiments, a FIX polypeptide as described herein is conjugated to a HEP polymer as described herein. Any FIX polypeptide as described herein may be combined with any HEP polymer as described herein

Common methods for linking half-life extending moieties such as carbohydrate polymers to glycoproteins comprise oxime, hydrazone or hydrazide bond formation. WO2006094810 describes methods for attaching hydroxyethyl starch polymers to glycoproteins such as erythropoietin that circumvent the problems connected to using activated ester chemistry. In these methods, hydroxyethyl starch and erythropoietin are individually oxidized with periodate on the carbohydrate moieties, and the reactive carbonyl groups ligated together using bis-hydroxylamine linking agents. The method will create hydroxyethyl starch linked to the erythropoietin via oxime bonds.

Similar oxime based linking methodology can be imagined for attaching carbohydrate polymers to GSC (cf. WO2011101267), however, as such oxime bonds are known to exist in both syn- and anti-isomer forms, the linkage between the polymer and the protein will contain both syn- and anti-isomer combinations. Such isomer mixtures are usually not desirable in proteinaceous medicaments that are used for long term repeating administration since the linker inhomogeneity may pose a risk for antibody generation.

The above mentioned methods have further disadvantages. In the oxidative process required for activating the glycoprotein, parts of the carbohydrate residues are chemically cleaved and the carbohydrates will therefore not be present in an intact form in the final conjugate. The oxidative process will, furthermore, generate product heterogenicity as the oxidating agent i.e. periodate in most cases is unspecific with regard to which glycan residue is oxidized. Both product heterogenicity and the presence of non-intact glycan residues in the final drug conjugate may impose immunogenicity risks.

Alternatives for linking carbohydrate polymers to glycoproteins involve the use of maleimide chemistry (WO2006094810). For example, the carbohydrate polymer can be furnished with a maleimido group, which selectively can react with a sulfhydryl group on the target protein. The linkage will then contain a cyclic succinimide group.

It is possible to link a carbohydrate polymer such as HEP via a maleimido group to a thio-modified GSC molecule and transfer the reagent to an intact glycosyl groups on a glycoprotein by means of a sialyltransferase, thereby creating a linkage that contains a cyclic succinimide group. Succinimide based linkages, however, may undergo hydrolytic ring opening when the conjugate is stored in aqueous solution for extended time periods (Bioconjugation Techniques, G. T. Hermanson, Academic Press, 3^{rd} edition 2013 p. 309) and while the linkage may remain intact, the ring opening reaction will add undesirable heterogeneity in form of regio- and stereo-isomers to the final conjugate.

It follows from the above that it is preferable to link the half-life extending moiety to the glycoprotein in such a way that 1) the glycan residue of the glycoprotein is preserved in intact form, and 2) no heterogenicity is present in the linker part between the intact glycosyl residue and the half-life extending moiety.

There is a need in the art for methods of conjugating a half-life extending moiety such as HEP to a protein glycan such as a FIX polypeptide glycan, wherein the compounds are linked such that a stable and isomer free conjugate is obtained.

In some embodiments a stable and isomer free linker is provided for use in sialic acid based conjugation of HEP to FIX wherein the HEP polymer may be attached to the sialic acid at positions appropriate for derivatization. Appropriate sites are known to the skilled person, or can be deduced from WO03031464 (which is hereby incorporated by reference in its entirety), wherein PEG polymers are attached to sialic acid cytidine monophosphate in multiple ways

In some embodiments the C4 and C5 position of the sialic acid pyranose ring, as well as the C7, C8 and C9 position of the side chain can serve as points of derivatization. Derivatization preferably involves the existing hetero atoms of the sialic acid, such as the hydroxyl or amine group, but functional group conversion to render appropriate attachment points on the sialic acid is also a possibility.

In some embodiments, the 9-hydroxy group of the sialic acid N-acetylneuraminic acid may be converted to an amino group by methods known in the art (Eur J Biochem 168, 594-602 (1987). The resulting 9-deoxy-amino N-acetylneuraminic acid cytidine monophosphate as shown below is an activated sialic acid derivative that can serve as an alternative to GSC.

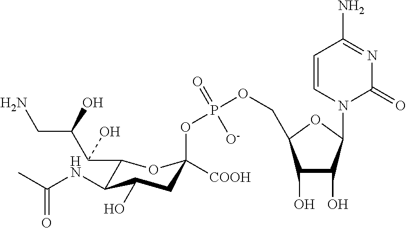

In some embodiments non-amine containing sialic acids such as 2-keto-3-deoxy-nonic acid, also known as KDN may also be converted to 9-amino derivatized sialic acids following same scheme.

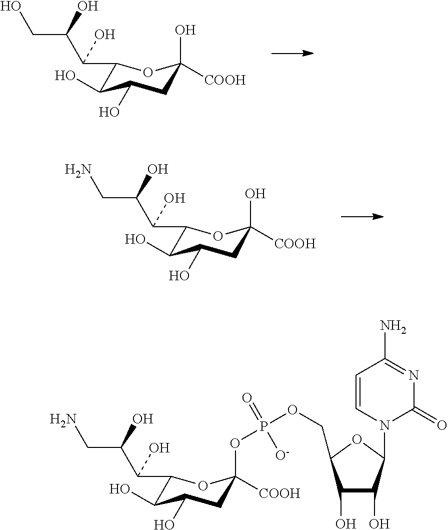

A similar scheme can be used for the shorter C8-sugar analogues belonging to the sialic acid family. Thus shorter versions of sialic acids such as 2-keto-3-deoxyoctonate, also known as KDO may be converted to the 8-deoxy-8-amino-2-keto-3-deoxyoctonate cytidine monophosphate, and used as an alternative to sialic acids that do not lack the C9 carbon atom.

In some embodiments, neuraminic acid cytidine monophosphate may be used in the invention. This material can be prepared as described in Eur J Org Chem. 2000, 1467-1482.

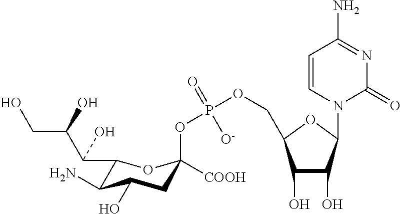

In some embodiments a stable and isomer free linker for use in glycyl sialic acid cytidine monophosphate (GSC) based conjugation of HEP to FIX is provided. The GSC starting material used in the current invention can be synthesised chemically (Dufner, G. Eur. J Org Chem 2000, 1467-1482) or it can be obtained by chemoenzymatic routes as described in WO2007056191. The GSC structure is shown below:

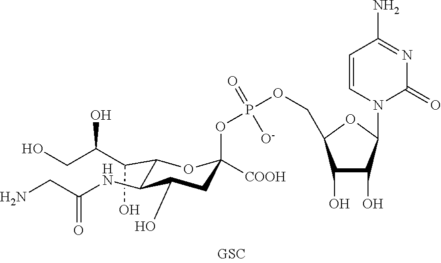

In some embodiments the conjugates described herein comprise a linker comprising the following structure:

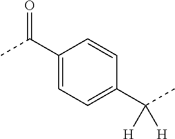

hereinafter also referred to as sublinker or sublinkage—that connects a HEP-amine and GSC in one of the following ways:

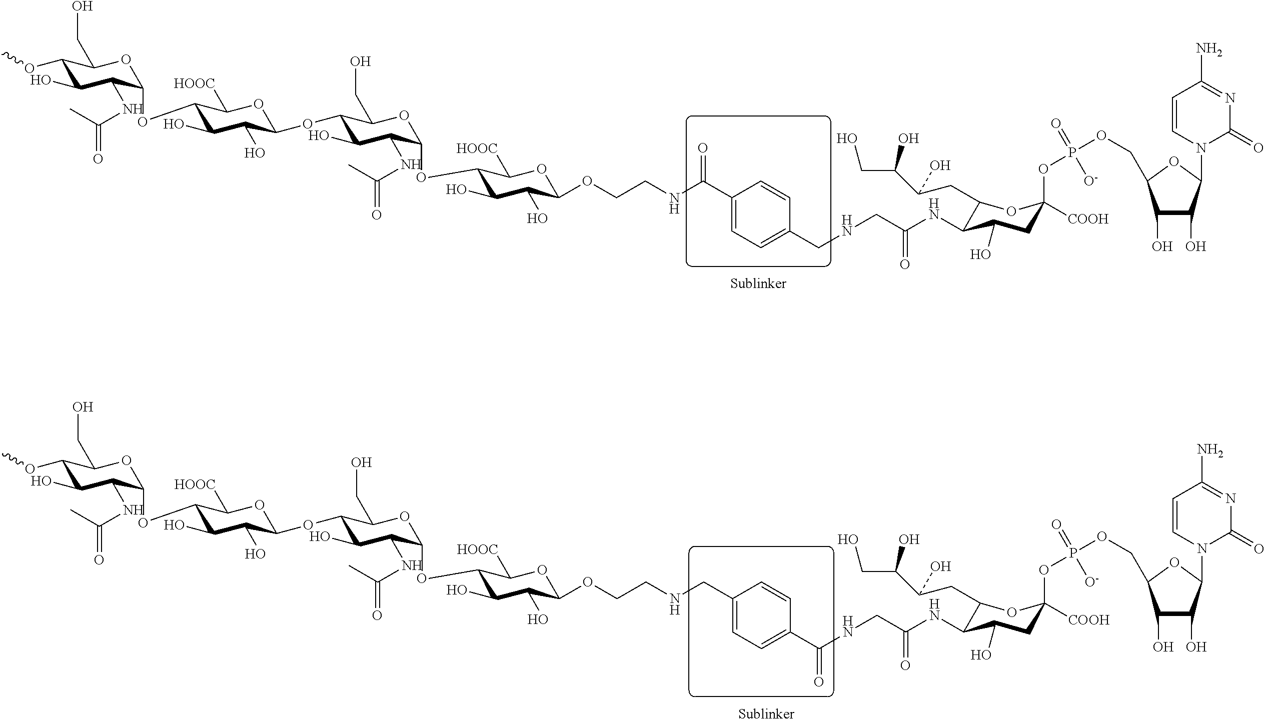

The highlighted 4-methylbenzoyl sublinker thus makes up part of the full linking structure linking the half-life extending moiety to a target protein. The sublinker is as such a stable structure compared to alternatives, such as succinimide based linkers (prepared from maleimide reactions with sulfhydryl groups) since the latter type of cyclic linkage has a tendency to undergo hydrolytic ring opening when the conjugate is stored in aqueous solution for extended time periods (Bioconjugation Techniques, G. T. Hermanson, Academic Press, 3^{rd} edition 2013 p. 309). Even though the linkage in this case (e.g. between HEP and sialic acid on a glycoprotein) may remain intact, the ring opening reaction will add heterogeneity in form of regio- and stereo-isomers to the final conjugate composition.

One advantage associated with conjugates according to the present invention is thus that a homogenous composition is obtained, i.e. that the tendency of isomer formation due to linker structure and stability is significantly reduced. Another advantage is that the linker and conjugates according to the invention can be produced in a simple process, preferably a one-step process.

Isomers are undesirable since these can lead to a heterogeneous product and increase the risk for unwanted immune responses in humans.

The 4-methylbenzoyl sublinkage as used between HEP and GSC, as used in the methods described herein, is not able to form stereo- or regio isomers. In a non-limiting embodiment FIG. 5 shows HEP conjugated onto a biantenna N-glycan on Factor IX (pos. N157 or N167) using the 4-methylbenzoyl sublinkage of the present invention.

Processes for preparation of functional HEP polymers are described in US20100036001 which for example lists aldehyde-, amine- and maleimide functionalized HEP reagents. US20100036001 is hereby incorporated by reference in its entirety as if fully set forth herein. A range of other functionally modified HEP derivatives are available using similar chemistry. HEP polymers used in certain embodiments of the present invention are initially produced with a primary amine handle at the reducing terminal according to methods described in US20100036001. HEP polymers with a primary amine handle (HEP-NH₂) can for example be prepared as described in Sismey-Ragatz et al., 2007 J Biol Chem and U.S. Pat. No. 8,088,604. Briefly, a fusion of the E. coli maltose-binding protein with PmHS1 is used as the catalyst to elongate heparosan oligosaccharide acceptors with a free amine at the reducing terminus into longer chains with UDP-GlcNAc and UDP-GlcUA precursors. The acceptor synchronizes the reaction so all chains are the same length (quasi-monodisperse size distribution) and it also imparts the free amine group to the sugar chain for subsequent modification or coupling reactions

Amine functionalized HEP polymers (i.e. HEP having an amine-handle) prepared according US20100036001 can be converted into a HEP-benzaldehyde by reaction with N-succinimidyl 4-formylbenzoate and subsequently coupled to the glycylamino group of GSC by a reductive amination reaction. The resulting HEP-GSC product can subsequently be enzymatically conjugated to a glycoprotein using a sialyltransferase.

For example said amine handle on HEP can be converted into a benzaldehyde functionality by reaction with N-succinimidyl 4-formylbenzoate according to the below scheme:

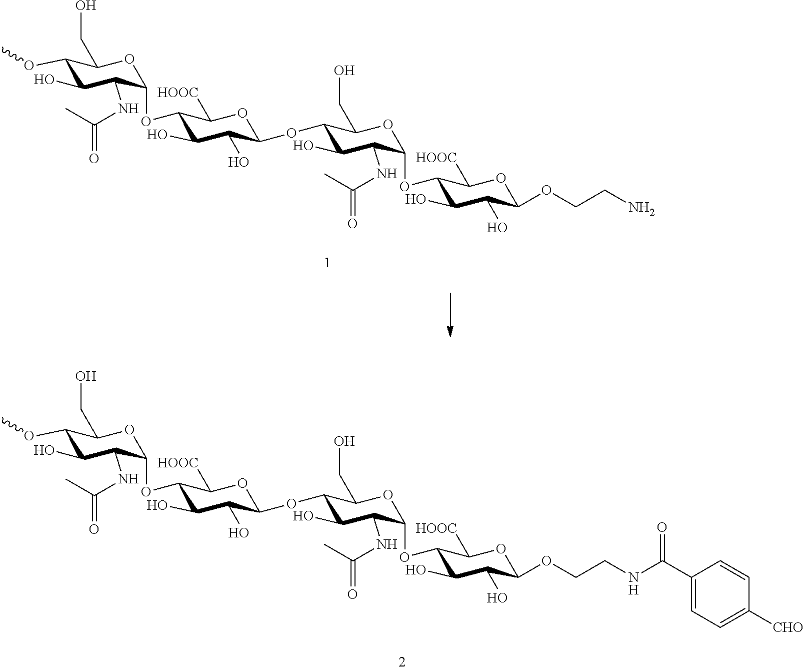

The conversion of HEP amine (1) to the 4-formylbenzamide compound (2) in the above scheme may be carried out by reaction with acyl activated forms of 4-formylbenzoic acid.

N-hydroxysuccinimidyl may be chosen as acyl activating group but a number of other acyl activation groups are known to the skilled person. Non-limited examples include 1-hydroxy-7-azabenzotriazole-, 1-hydroxy-benzotriazole-, pentafluorophenyl-esters as know from peptide chemistry.

HEP reagents modified with a benzaldehyde functionality can be kept stable for extended time periods when stored frozen (−80° C.) in dry form.

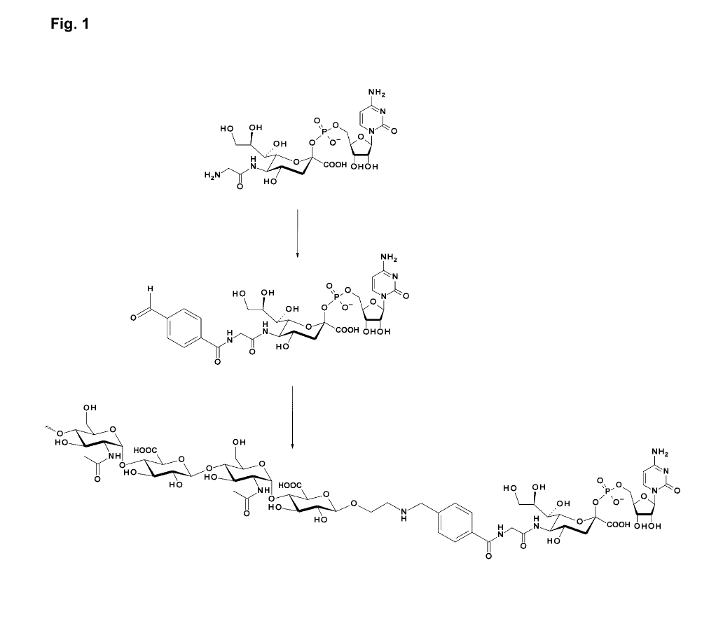

Alternatively, a benzaldehyde moiety can be attached to the GSC compound, thereby resulting in a GSC-benzaldehyde compound suitable for conjugation to an amine functionalized HEP moiety. This route of synthesis is depicted in FIG. 1.

For example, GSC can be reacted under pH neutral conditions with N-succinimidyl 4-formylbenzoate to provide a GSC compound that contains a reactive aldehyde group. The aldehyde derivatized GSC compound (GSC-benzaldehyde) can then be reacted with HEP-amine and reducing agent to form a HEP-GSC reagent.

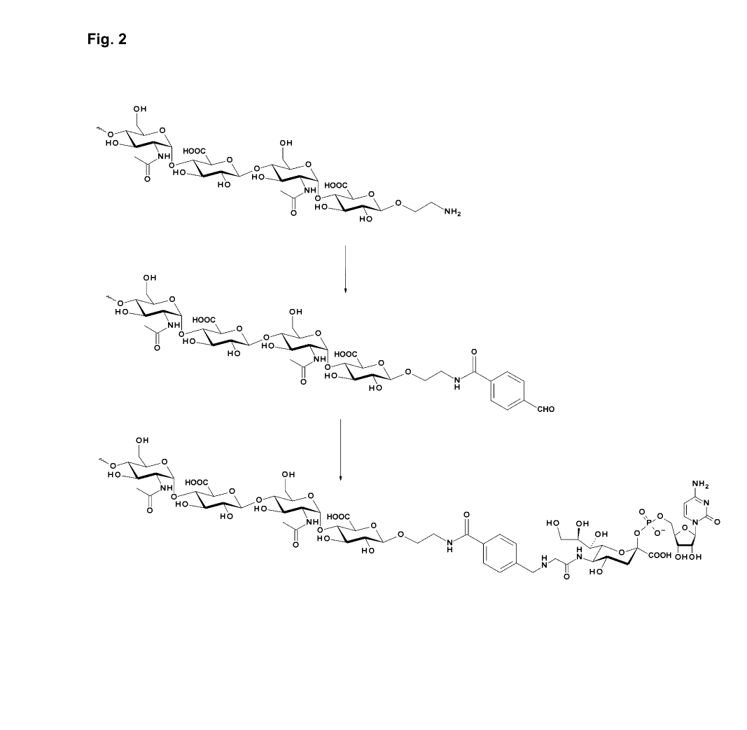

The above mentioned reaction may be reversed, so that the HEP-amine is first reacted with N-succinimidyl 4-formylbenzoate to form an aldehyde derivatized HEP-polymer, which subsequently is reacted directly with GSC in the presence of a reducing agent. In practice this eliminates the tedious chromatographic handling of GSC-CHO. This route of synthesis is depicted in FIG. 2.

Thus, in some embodiments, HEP-benzaldehyde is coupled to GSC by reductive amination.

Reductive amination is a two-step reaction which proceeds as follows: Initially an imine (also known as Schiff-base) is formed between the aldehyde component and the amine component (in the present embodiment the glycyl amino group of GSC). The imine is then reduced to an amine in the second step. The reducing agent is chosen so that it selectively reduces the formed imine to an amine derivative.

A number of suitable reducing reagents are available to the skilled person. Non-limiting examples include sodium cyanoborohydride (NaBH3CN), sodium borohydride (NaBH4), pyridin boran complex (BH3:Py), dimethylsulfide boran complex (Me2S:BH3) and picoline boran complex.

Although reductive amination to the reducing end of carbohydrates (for example to the reducing termini of HEP polymers) is possible, it has generally been described as a slow and inefficient reaction (J C. Gildersleeve, Bioconjug Chem. 2008 July; 19(7): 1485-1490). Side reactions, such as the Amadori reaction, where the initially formed imine rearrange to a keto amine are also possible, and will lead to heterogenicity which as previously discussed is undesirable in the present context.

Aromatic aldehydes such as benzaldehydes derivatives are not able to form such rearrangement reactions as the imine is unable to enolize and also lack the required neighbouring hydroxy group typically found in carbohydrate derived imines. Aromatic aldehydes such as benzaldehydes derivatives are therefore particular useful in reductive amination reactions for generating the isomer free HEP-GSC reagent.

A surplus of GSC and reducing reagent is optionally used in order to drive reductive amination chemistry fast to completion. When the reaction is completed, the excess (non-reacted) GSC reagent and other small molecular components such as excess reducing reagent can subsequently be removed by dialysis, tangential flow filtration or size exclusion chromatography.

Both the natural substrate for sialyltransferases, Sia-CMP, and the GSC derivatives are multifunctional molecules that are charged and highly hydrophilic. In addition, they are not stable in solution for extended time periods especially if pH is below 6.0. At such low pH, the CMP activation group necessary for substrate transfer is lost due to acid catalyzed phosphate diester hydrolysis (Yasuhiro Kajihara et al., Chem Eur J 2011, 17, 7645-7655). Selective modification and isolation of GSC and Sia-CMP derivatives thus require careful control of pH, as well as fast and efficient isolation methods, in order to avoid CMP-hydrolysis.

In some embodiments, large half-life extending moieties are conjugated to GSC using reductive amination chemistry. Arylaldehydes, such as benzaldehyde modified HEP polymers have been found optimal for this type of modification, as they can efficiently react with GSC under reductive amination conditions.

As GSC may undergo hydrolysis in acid media, it is important to maintain a near neutral or slightly basic environment during the coupling to HEP-benzaldehyde. HEP polymers and GSC are both highly water soluble and aqueous buffer systems are therefore preferable for maintaining pH at a near neutral level. A number of both organic and inorganic buffers may be used; however, the buffer components should preferably not be reactive under reductive amination conditions. This excludes for instance organic buffer systems containing primary and—to a lesser extent—secondary amino groups. Informed by the present description, the skilled person will know which buffers are suitable and which are not. Some examples of suitable buffers are shown in Table 1 below:

TABLE 1
Buffers

| Common | pKa at | Buffer | |
|---|---|---|---|
| Name | 25° C. | Range | Full Compound Name |
| Bicine | 8.35 | 7.6-9.0 | N,N-bis(2-hydroxyethyl)glycine |
| Hepes | 7.48 | 6.8-8.2 | 4-2-hydroxyethyl-1-piperazineethane- |
| | | | sulfonic acid |
| TES | 7.40 | 6.8-8.2 | 2-{[tris(hydroxymethyl)methyl]ami- |
| | | | no}ethanesulfonic acid |
| MOPS | 7.20 | 6.5-7.9 | 3-(N-morpholino)propanesulfonic acid |
| PIPES | 6.76 | 6.1-7.5 | Piperazine-N,N′-bis(2-ethanesulfonic acid) |
| MES | 6.15 | 5.5-6.7 | 2-(N-morpholino)ethanesulfonic acid |

By applying this method, GSC reagents modified with half-life extending moieties such as HEP, having isomer free stable linkages can be prepared efficiently, and isolated in a simple process that minimize the chance for hydrolysis of the CMP activation group.

By reacting either of said compounds with each other a HEP-GSC conjugate comprising a 4-methylbenzoyl sublinker moiety may be created.

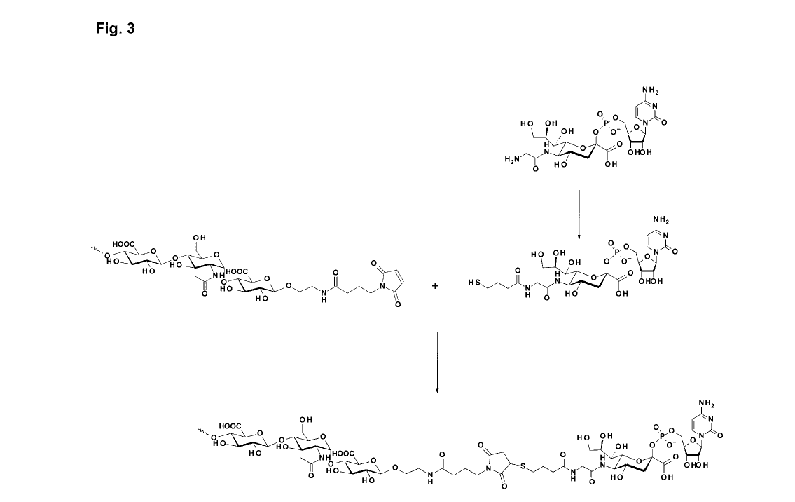

GSC may also be reacted with thiobutyrolactone, thereby creating a thiol modified GSC molecule (GSC-SH). Such reagents may be reacted with maleimide functionalized HEP polymers to form HEP-GSC reagents. This synthesis route is depicted in FIG. 3. The resulting product has a linkage structure comprising succinimide.

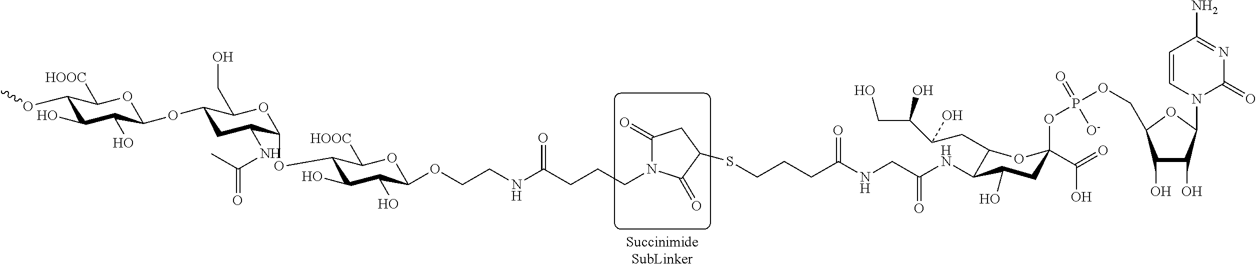

However, succinimide based (sub)linkages may undergo hydrolytic ring opening inter alia when the modified GSC reagent is stored in aqueous solution for extended time periods and while the linkage may remain intact, the ring opening reaction will add undesirable heterogeneity in form of regio- and stereo-isomers.

### Methods of Glycoconjugation

Conjugation of a HEP-GSC conjugate with a polypeptide may be carried out via a glycan present on residues in the polypeptide backbone. This form of conjugation is also referred to as glycoconjugation.

In contrast to conjugation methods based on cysteine alkylations, lysine acylations and similar conjugations involving amino acids in the protein backbone, conjugation via glycans is an appealing way of attaching larger structures such as a HEP polymer to bioactive proteins with less disturbance of bioactivity. This is because glycans being highly hydrophilic generally tend to be oriented away from the protein surface and out in solution, leaving the binding surfaces that are important for the proteins activity free.

The glycan may be naturally occurring or it may be inserted via e.g. insertion of an N-linked glycan using methods well known in the art.

Methods for glycoconjugation of HEP polymers include galactose oxidase based conjugation (WO2005014035) and periodate based conjugation (WO2008025856). Methods based on sialyltransferase have over the years proven to be mild and highly selective for modifying N-glycans or O-glcyans on blood coagulation factors, such as FIX.

GSC is a sialic acid derivative that can be transferred to glycoproteins by the use of sialyltransferases. It can be selectively modified with substituents such as PEG or HEP on the glycyl amino group and still be enzymatically transferred to glycoproteins by use of sialyltransferases. GSC can be efficiently prepared by an enzymatic process in large scale (WO2007056191).

In some embodiments, terminal sialic acids on FIX glycans are removed by sialidase treatment to provide asialoFIX. AsialoFIX and GSC modified with HEP together will act as substrates for sialyltransferases. The product of the sialyltransferase reaction is a HEP-FIX conjugate having HEP linked via an intact glycosyl linking group on the glycan.

### Sialyltransferases

Sialyltransferases are a class of glycosyltransferases that transfer sialic acid from naturally activated sialic acid (Sia)—CMP (cytidine monophosphate) compounds to galactosyl-moieties on e.g. proteins. Many sialyltransferases (ST3GaIIII, ST3GaII, ST6GalNAcl) are capable of transfer of sialic acid—CMP (Sia-CMP) derivatives that have been modified on the C5 acetamido group inter alia with large groups such as 40 kDa PEG (WO03031464). An extensive, but non-limited list of relevant sialyltransferases that can be used with the current invention is disclosed in WO2006094810, which is hereby incorporated by reference in its entirety.

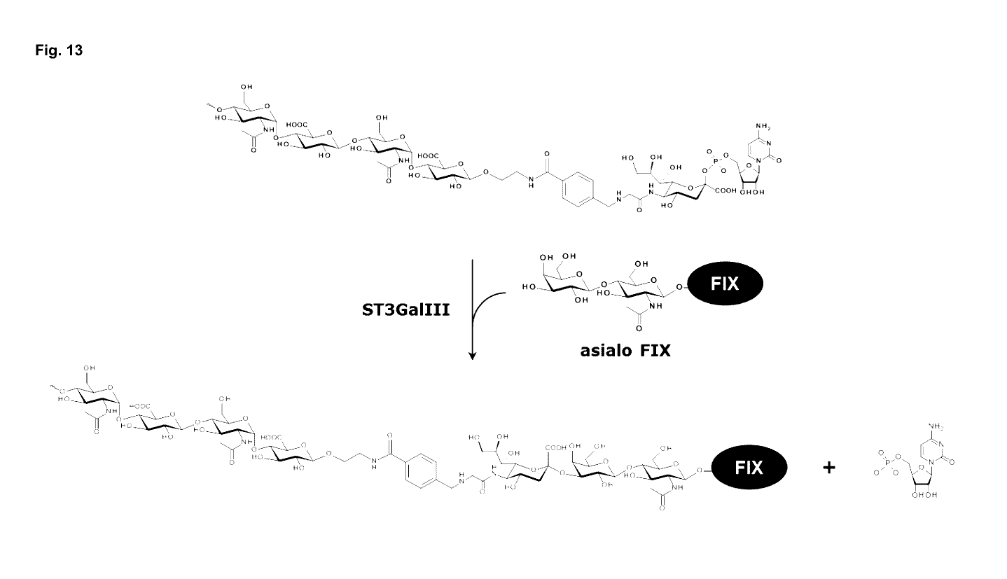

In some embodiments, terminal sialic acids on glycoproteins are removed by sialidase treatment to provide asialo glycoproteins. Asialo glycoproteins and GSC modified with the half-life extending moiety together will act as substrates for sialyltransferases. The product of the reaction is a glycoprotein conjugate having the half-life extending moiety linked via an intact glycosyl linking group—in this case an intact sialic acid linker group. A reaction scheme wherein an asialoFIX glycoprotein is reacted with HEP-GSC in the presence of sialyltransferase is shown in FIG. 13.

### Properties of HEP-FIX Conjugates

In some embodiments, the conjugates described herein have various advantageous biological properties. For example, the conjugate may show one of more of the following (non-limiting) advantages when compared to a suitable control FIX molecule:
improved circulation half-life in vivo
improved mean residence time in vivo
improved biodegradability in vivo
improved bleeding time and blood loss in a tail vein transection (TVT) model in FIX knock-out mice
improved inter-assay variability in various aPTT-based assays

The conjugate may show an improvement in any biological activity of FIX as described herein and this may be measured using any assay or method as described herein, such as the methods described below in relation to the activity of FIX.

Advantages may be seen when a conjugate of the invention is compared to a suitable control FIX molecule. The control molecule may be, for example, an unconjugated FIX polypeptide or a conjugated FIX polypeptide. The conjugated control may be a FIXa polypeptide conjugated to a water soluble polymer, or a FIXa polypeptide chemically linked to a protein. A conjugated FIX control may be a FIX polypeptide that is conjugated to a chemical moiety (being protein or water soluble polymer) of a similar size as the HEP molecule in the conjugate of interest. The water-soluble polymer can for example be PEG, branched PEG, dextran, poly(1-hydroxymethylethylene hydroxymethylformal) or 2-methacryloyloxy-2′-ethyltrimethylammoniumphosphate (MPC).

The FIX polypeptide in the control FIX molecule is preferably the same FIX polypeptide that is present in the conjugate of interest. For example, the control FIX molecule may have the same amino acid sequence as the FIX polypeptide in the conjugate of interest. The control FIX may have the same glycosylation pattern as the FIX polypeptide in the conjugate of interest.

In some embodiments, conjugates as described herein have an improvement in circulatory half-life, or in mean residence time when compared to a suitable control.

In some embodiments, conjugates as described herein have a modified circulatory half-life compared to the wild type protein molecule, preferably an increased circulatory half-life. Circulatory half-life is preferably increased at least 10%, preferably at least 15%, preferably at least 20%, preferably at least 25%, preferably at least 30%, preferably at least 35%, preferably at least 40%, preferably at least 45%, preferably at least 50%, preferably at least 55%, preferably at least 60%, preferably at least 65%, preferably at least 70%, preferably at least 75%, preferably at least 80%, preferably at least 85%, preferably at least 90%, preferably at least 95%, preferably at least 100%, more preferably at least 125%, more preferably at least 150%, more preferably at least 175%, more preferably at least 200%, and most preferably at least 250% or 300%. Even more preferably, such molecules have a circulatory half-life that is increased at least 400%, 500%, 600%, or even 700%.

Where the activity being compared is a biological activity of FIX, such as clotting activity or proteolysis, the control can be a suitable FIX polypeptide conjugated to a water soluble polymer of comparable size to the HEP conjugate of the current invention.

The conjugate may not retain the level of biological activity seen in FIX that is not modified by the addition of HEP. Preferably, the conjugate retains as much of the biological activity of unconjugated FIX as possible. For example, the conjugate may retain at least 15%, at least 20%, at least 25%, at least 30%, at least 35%, at least 40%, at least 45%, at least 50%, at least 60%, at least 70%, at least 80% or at least 90% of the biological activity of an unconjugated FIX control. As discussed above, the control may be a FIX molecule having the same amino acid sequence as the FIX polypeptide in the conjugate, but lacking HEP. The conjugate may, however, show an improvement in biological activity when compared to a suitable control. The biological activity here may be any biological activity of FIX as described herein such as clotting activity or proteolysis activity.

An improved biological activity when compared to a suitable control as described herein may be any measurable or statistically significant increase in a biological activity. The biological activity may be any biological activity of FIX as described herein, such as clotting activity, proteolytic activity, reduction of bleeding time and blood loss. The increase may be, for example, an increase of at least 5%, at least 10%, at least 15%, at least 20%, at least 25%, at least 30%, at least 35%, at least 40%, at least 45%, at least 50%, at least 55%, at least 60%, at least 70% or more in the relevant biological activity when compared to the same activity in a suitable control.

An advantage of the conjugates as described herein is that HEP polymers are enzymatically biodegradable. The conjugates are therefore preferably enzymatically degradable in vivo.

In some embodiments the conjugates comprising a HEP polymer linked to FIX reduces or not cause significant inter-assay variability in when using different aPTT-based clotting assays.

### Compositions

In another aspect, the present invention provides compositions comprising conjugates as described herein. In some embodiments the pharmaceutical composition comprises one or more conjugates formulated together with a pharmaceutically acceptable carrier.

As used herein, “pharmaceutically acceptable carrier” includes any and all solvents, dispersion media, coatings, antibacterial and antifungal agents, isotonic and absorption delaying agents, and the like that are physiologically compatible.

Preferred pharmaceutically acceptable carriers comprise aqueous carriers or diluents. Examples of suitable aqueous carriers that may be employed in the pharmaceutical compositions of the invention include water, buffered water and saline. Examples of other carriers include ethanol, polyols (such as glycerol, propylene glycol, PEG, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants. In many cases, it will be preferable to include isotonic agents, for example, sugars, polyalcohols such as mannitol, sorbitol, or sodium chloride in the composition.

The pharmaceutical compositions are primarily intended for parenteral administration for prophylactic and/or therapeutic treatment. Preferably, the pharmaceutical compositions are administered parenterally, i.e., intravenously, subcutaneously, or intramuscularly, or it may be administered by continuous or pulsatile infusion. The compositions for parenteral administration comprise the FIX conjugate of the invention in combination with, preferably dissolved in, a pharmaceutically acceptable carrier, preferably an aqueous carrier. A variety of aqueous carriers may be used, such as water, buffered water, 0.9% saline, 0.4% saline, 0.3% glycine and the like. The FIX conjugates as described herein can also be formulated into liposome preparations for delivery or targeting to the sites of injury. Liposome preparations are generally described in, e.g., U.S. Pat. No. 4,837,028, U.S. Pat. No. 4,501,728 and U.S. Pat. No. 4,975,282. The compositions may be sterilised by conventional, well-known sterilisation techniques. The resulting aqueous solutions may be packaged for use or filtered under aseptic conditions and lyophilised, the lyophilised preparation being combined with a sterile aqueous solution prior to administration. The compositions may contain pharmaceutically acceptable auxiliary substances as required to approximate physiological conditions, such as pH adjusting and buffering agents, tonicity adjusting agents and the like, for example, sodium acetate, sodium lactate, sodium chloride, potassium chloride, calcium chloride, etc.

The concentration of FIX conjugate in these formulations can vary widely, i.e., from less than about 0.5% by weight, usually at or at least about 1% by weight to as much as 15 or 20% by weight and will be selected primarily by fluid volumes, viscosities, etc., in accordance with the particular mode of administration selected. Actual methods for preparing parenterally administrable compositions will be known or apparent to those skilled in the art and are described in more detail in, for example, Remington's Pharmaceutical Sciences, 18th ed., Mack Publishing Company, Easton, Pa. (1990).

The composition can be formulated as a solution, microemulsion, liposome, or other ordered structure suitable to high drug concentration.

The composition should be sterile and should be fluid to the extent that easy syringability exists. The composition should be stable under the conditions of manufacture and storage and may be preserved against the contaminating action of microorganisms such as bacteria and fungi. In the case of sterile powders for the preparation of sterile injectable solutions, the preferred methods of preparation are vacuum drying and freeze-drying (lyophilization) that yield a powder of the active agent plus any additional desired ingredient from a previously sterile-filtered solution thereof.

Prevention of the action of microorganisms may be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions may be brought about by including in the composition an agent that delays absorption, for example, aluminium monostearate or gelatin.

Sterile injectable solutions may be prepared by incorporating the conjugates as described herein in the required amount in an appropriate solvent with one or a combination of ingredients enumerated above, as required, followed by filtered sterilization. Generally, dispersions are prepared by incorporating the conjugate into a sterile carrier that contains a basic dispersion medium and the required other ingredients from those enumerated above. In the case of sterile powders for the preparation of sterile injectable solutions, the methods of preparation may include vacuum drying, spray drying, spray freezing and freeze-drying that yields a powder of the active ingredient (i.e., the HEP conjugate) plus any additional desired ingredient from a previously sterile-filtered solution thereof.

Compositions may be formulated in dosage unit form for ease of administration and uniformity of dosage. Dosage unit form as used herein refers to physically discrete units suited as unitary dosages for the subjects to be treated; each unit containing a predetermined quantity of conjugate calculated to produce the desired therapeutic effect. The specification for the dosage unit forms of the presently claimed and disclosed invention(s) are dictated by and directly dependent on (a) the unique characteristics of the HEP conjugate and the particular therapeutic effect to be achieved, and (b) the limitations inherent in the art of compounding such a therapeutic compound for the treatment of a selected condition in a subject.

Pharmaceutical compositions as described herein may comprise additional active ingredients in addition to a conjugate as described herein. For example, a pharmaceutical composition may comprise additional therapeutic or prophylactic agents. For example, where a pharmaceutical composition is intended for use in the treatment of a bleeding disorder, it may additionally comprise one or more agents intended to reduce the symptoms of the bleeding disorder. For example, the composition may comprise one or more additional clotting factors. The composition may comprise one or more other components intended to improve the condition of the patient. For example, where the composition is intended for use in the treatment of patients suffering from unwanted bleeding such as patients undergoing surgery or patients suffering from trauma, the composition may comprise one or more analgesic, anaesthetic, immunosuppressant or anti-inflammatory agents.

The composition may be formulated for use in a particular method or for administration by a particular route. A conjugate or composition of the invention may be administered parenterally, intraperitoneally, intraspinally, intravenously, intramuscularly, intravaginally, subcutaneously, intranasally, rectally, or intracerebrally.

An advantageous property of the HEP-FIX polypeptide conjugates as described herein is where the polymer has a polymer size around in the range of 13 to 65 kDa (in particular 13 to 55 kDa, 13 to 50 kDa, 13 to 45 kDa, 13 to 40 kDa, 25 to 55 kDa, 25 to 50 kDa, 25 to 45 kDa, 30 to 45 kDa or 38 to 42 kDa) as this may allow for an in vivo useful half-life or mean residence time while also having a suitable viscosity in liquid solution.

### Uses of the Conjugates

Conjugates as described herein may be administered to an individual in need thereof in order to deliver FIX polypeptides to that individual. The individual may be any individual in need of FIX polypeptides.

The FIX polypeptides conjugates according to the present invention may be used to control bleeding disorders which may be caused by, for example, clotting factor deficiencies (e.g. haemophilia B) or clotting factor inhibitors, or they may be used to control excessive bleeding occurring in subjects with a normally functioning blood clotting cascade (no clotting factor deficiencies or inhibitors against any of the coagulation factors).

For treatment in connection with deliberate interventions, the FIX polypeptide conjugates of the invention will typically be administered within about 24 hours—or even earlier due to prolonged half-life—prior to performing the intervention, and for as much as 7 days or more thereafter. Administration can be carried out by a variety of routes as described herein.

The dose of the FIX polypeptide delivered may be from about 0.05 mg to 500 mg of the FIX polypeptide conjugate per day, preferably from about 1 mg to 100 mg per day, and more preferably from about 5 mg to about 75 mg per day for a 70 kg subject as loading and maintenance doses, depending on the severity of the condition. A suitable dose may also be adjusted for a particular conjugate of the invention based on the properties of that conjugate, including its in vivo half-life or mean residence time and its biological activity. For example, conjugates having a longer half-life may be administered in reduced dosages and/or compositions having reduced activity compared to wild-type FIX may be administered in increased dosages.

The compositions containing the FIX polypeptide conjugates of the present invention can be administered for prophylactic and/or therapeutic treatments. In therapeutic applications, compositions are administered to a subject already suffering from a disease, such as any bleeding disorder as described above, in an amount sufficient to cure, alleviate or partially arrest the disease and its complications. An amount adequate to accomplish this is defined as “therapeutically effective amount”. As will be understood by the person skilled in the art amounts effective for this purpose will depend on the severity of the disease or injury as well as the weight and general state of the subject. In general, however, the effective delivery amount will range from about 0.05 mg up to about 500 mg of the FIX polypeptide conjugate per day for a 70 kg subject, with dosages of from about 1.0 mg to about 100 mg of the conjugate being delivered per day being more commonly used.

The conjugates as described herein may generally be employed in serious disease or injury states, that is, life threatening or potentially life threatening situations. In such cases, in view of the minimisation of extraneous substances and general lack of immunogenicity of human FIX polypeptide variants in humans, it may be felt desirable by the treating physician to administer a substantial excess of these FIX conjugate compositions. In prophylactic applications, compositions containing the FIX conjugate of the invention are administered to a subject susceptible to or otherwise at risk of a disease state or injury to enhance the subject's own coagulative capability. Such an amount is defined to be a “prophylactically effective dose.” In prophylactic applications, the precise amounts of FIX polypeptide conjugate being delivered once again depend on the subject's state of health and weight, but the dose generally ranges from about 0.05 mg to about 500 mg per day for a 70 kg subject, more commonly from about 1.0 mg to about 100 mg per day for a 70 kg subject.

Single or multiple administrations of the compositions can be carried out with dose levels and patterns being selected by the treating physician. For ambulatory subjects requiring daily maintenance levels, the FIX polypeptide conjugates may be administered by continuous infusion using e.g. a portable pump system.

Local delivery of a FIX conjugate of the present invention, such as, for example, topical application may be carried out, for example, by means of a spray, perfusion, double balloon catheters, stent, incorporated into vascular grafts or stents, hydrogels used to coat balloon catheters, or other well established methods. In any event, the pharmaceutical compositions should provide a quantity of FIX polypeptide conjugate sufficient to effectively treat the subject.

### DEFINITIONS

Unless defined otherwise, all technical and scientific terms used herein generally have the same meaning as commonly understood by one of ordinary skill in the art to which this, invention belongs.

The term “subject”, as used herein, includes any human patient, or non-human vertebrate.

The term “treatment” refers to the medical therapy of any human or other vertebrate subject in need thereof. Said subject is expected to have undergone physical examination by a medical practitioner, or a veterinary medical practitioner, who has given a tentative or definitive diagnosis which would indicate that the use of said specific treatment is beneficial to the health of said human or other vertebrate. The timing and purpose of said treatment may vary from one individual to another, according to the status quo of the subject's health. Thus, said treatment may be prophylactic, palliative, symptomatic and/or curative. In terms of the present invention, prophylactic, palliative, symptomatic and/or curative treatments may represent separate aspects of the invention.

The term “coagulopathy” refers to an increased haemorrhagic tendency which may be caused by any qualitative or quantitative deficiency of any pro-coagulative component of the normal coagulation cascade, or any upregulation of fibrinolysis. Such coagulopathies may be congenital and/or acquired and/or iatrogenic and are identified by a person skilled in the art.

The term “glycan” refers to the entire oligosaccharide structure that is covalently linked to a single amino acid residue. Glycans are normally N-linked or O-linked, e.g., glycans are linked to an asparagine residue (N-linked glycosylation) or a serine or threonine residue (O-linked glycosylation). N-linked oligosaccharide chains may be multi-antennary, such as, e.g., bi-, tri, or tetra-antennary and most often contain a core structure of Man3-GlcNAc-GlcNAc-. Both N-glycans and O-glycans are attached to proteins by the cells producing the protein. The cellular N-glycosylation machinery recognizes and glycosylates N-glycosylation consensus motifs (N—X—S/T motifs) in the amino acid chain, as the nascent protein is translocated from the ribosome to the endoplasmic reticulum (Kiely et al. (1976) Journal of Biological Chemistry 251, 5490-5495; Glabe et al. (1980) Journal of Biological Chemistry 255, 9236-9242). Some glycoproteins, when produced in a human in situ, have a glycan structure with terminal, or “capping”, sialic acid residues, i.e., the terminal sugar of each antenna is N-acetylneuraminic acid linked to galactose via an a2->3 or a2->6 linkage. Other glycoproteins have glycans end-capped with other sugar residues. When produced in other circumstances, however, glycoproteins may contain oligosaccharide chains having different terminal structures on one or more of their antennae, such as, e.g., containing N-glycolylneuraminic acid (Neu5Gc) residues or containing a terminal N-acetylgalactosamine (GalNAc) residue in place of galactose.

The term “half-life” as used herein in the context of administering a peptide drug to a patient refers to the time required for plasma concentration of a drug in a patient to be reduced by one half.

The term “half-life extending moiety” refers to one or more chemical groups that can increase in vivo circulation half-life of a number of therapeutic proteins/peptides when conjugated to these proteins/peptides. Examples of half-life extending moieties include: biocompatible fatty acids and derivatives thereof, Hydroxy Alkyl Starch (HAS) e.g. Hydroxy Ethyl Starch (HES), Poly Ethylene Glycol (PEG) and any combination thereof.

The term “recovery of Factor IX activity” refers to the activity measured in the aPTT assay in percent of the activity measured using the chromogenic assay.

The term “sialic acid” refers to any member of a family of nine-carbon carboxylated sugars. The most common member of the sialic acid family is N-acetylneuraminic acid (2-keto-5-acetamido-3,5-dideoxy-D-glycero-D-galactononulopyranos-1-onic acid (often abbreviated as Neu5Ac, NeuAc, NeuNAc, or NANA). A second member of the family is N-glycolyl-neuraminic acid (Neu5Gc or NeuGc), in which the N-acetyl group of NeuNAc is hydroxylated. A third sialic acid family member is 2-keto-3-deoxy-nonulosonic acid (KDN) (Nadano et al. (1986) J Biol Chem 261: 11550-11557; Kanamori et al., J Biol Chem 265: 21811-21819 (1990)). Also included are 9-substituted sialic acids such as a 9-O—C1-C6 acyl-Neu5Ac like 9-O-lactylNeu5Ac or 9-O-acetyl-Neu5Ac. The synthesis and use of sialic acid compounds in a sialylation procedure is disclosed in international application WO92/16640, published Oct. 1, 1992.

The term “sialic acid derivative” refers to a sialic acid as defined above that is modified with one or more chemical moieties. The modifying group may for example be alkyl groups such as methyl groups, azido- and fluoro groups, or functional groups such as amino or thiol groups that can function as handles for attaching other chemical moieties. Examples include 9-deoxy-9-fluoro-Neu5Ac and 9-azido-9-deoxy-Neu5Ac. The term also encompasses sialic acids that lack one of more functional groups such as the carboxyl group or one or more of the hydroxyl groups. Derivatives where the carboxyl group is replaced with a carboxamide group or an ester group are also encompassed by the term. The term also refers to sialic acids where one or more hydroxyl groups have been oxidized to carbonyl groups. Furthermore the term refers to sialic acids that lack the C9 carbon atom or both the C9-C8 carbon chain for example after oxidative treatment with periodate.

Glycyl sialic acid is a sialic acid derivative according to the definition above, where the N-acetyl group of NeuNAc is replaced with a glycyl group also known as an amino acetyl group. Glycyl sialic acid may be represented with the following structure:

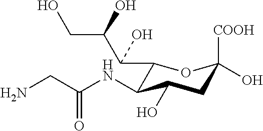

The term “CMP-activated” sialic acid or sialic acid derivatives refer to a sugar nucleotide containing a sialic acid moiety and a cytidine monophosphate (CMP).

In the present description, the term “glycyl sialic acid cytidine monophosphate” is used for describing GSC, and is a synonym for alternative naming of same CMP activated glycyl sialic acid. Alternative naming include CMP-5′-glycyl sialic acid, cytidine-5′-monophospho-N-glycylneuraminic acid, cytidine-5′-monophospho-N-glycyl sialic acid.

The term “intact glycosyl linking group” refers to a linking group that is derived from a glycosyl moiety in which the saccharide monomer interposed between and covalently attached to the polypeptide and the HEP moiety is not degraded, e.g., oxidized, e.g., by sodium metaperiodate during conjugate formation. “Intact glycosyl linking groups” may be derived from a naturally occurring oligosaccharide by addition of glycosyl units or removal of one or more glycosyl unit from a parent saccharide structure.

The term “asialo glycoprotein” is intended to include glycoproteins wherein one or more terminal sialic acid residues have been removed, e.g., by treatment with a sialidase or by chemical treatment, exposing at least one galactose or N-acetylgalactosamine residue from the underlying “layer” of galactose or N-acetylgalactosamine (“exposed galactose residue”).

Dotted lines in structure formulas denotes open valence bond (i.e. bonds that connect the structures to other chemical moieties).

### FURTHER EMBODIMENTS

In one embodiment the FIX polypeptide conjugated to HEP is wild type FIX.

In one embodiment the FIX polypeptide conjugated to HEP is wild type FIX(a).

In another embodiment the FIX polypeptide conjugated to HEP is an analogue or variant having >95% sequence identity to wild-type FIX or FIX(a).

In one embodiment the FIX of the HEP-FIX polypeptide conjugate is mutated so that it has increased proteolytic activity.

In one embodiment the HEP polymer conjugated to the FIX polypeptide has a molecular weight of 5 to 15 kDa.

In one embodiment the HEP polymer conjugated to the FIX polypeptide has a molecular weight of 15 to 25 kDa.

In one embodiment the HEP polymer conjugated to the FIX polypeptide has a molecular weight of 25 to 35 kDa.

In one embodiment the HEP polymer conjugated to the FIX polypeptide has a molecular weight of 35 to 45 kDa.

In one embodiment the HEP polymer conjugated to the FIX polypeptide has a molecular weight of 45 to 55 kDa.

In one embodiment the HEP polymer conjugated to the FIX polypeptide has a molecular weight of 55 to 65 kDa.

In one embodiment the HEP polymer conjugated to the FIX polypeptide has a molecular weight of 13 to 60 kDa.

In one embodiment the HEP polymer conjugated to the FIX polypeptide has a molecular weight of 13 to 50 kDa.

In one embodiment the HEP polymer conjugated to the FIX polypeptide has a molecular weight of 13 to 45 kDa.

In one embodiment the HEP polymer conjugated to the FIX polypeptide has a molecular weight of 13 to 40 kDa.

In one embodiment the HEP polymer conjugated to the FIX polypeptide has a molecular weight of 13 to 35 kDa.

In one embodiment the HEP polymer conjugated to the FIX polypeptide has a molecular weight of 13 to 30 kDa.

In one embodiment the HEP polymer conjugated to the FIX polypeptide has a molecular weight of 13 to 25 kDa.

In one embodiment the HEP polymer conjugated to the FIX polypeptide has a molecular weight of 27 to 40 kDa.

In one embodiment the HEP polymer conjugated to the FIX polypeptide has a molecular weight of 27 to 41 kDa.

In one embodiment the HEP polymer conjugated to the FIX polypeptide has a molecular weight of 27 to 42 kDa.

In one embodiment the HEP polymer conjugated to the FIX polypeptide has a molecular weight of 27 to 43 kDa.

In one embodiment the HEP polymer conjugated to the FIX polypeptide has a molecular weight of 27 to 44 kDa.

In one embodiment the HEP polymer conjugated to the FIX polypeptide has a molecular weight of 27 to 45 kDa.

In one embodiment, the HEP polymer has a size of about 5, 10, 15, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 80, 85, 90, 95, 100, 110, 120, 130, 140, 150, 160, 170, 175, 180, 190, or 200 kDa.

In one preferred embodiment the HEP polymer conjugated to the FIX polypeptide has a molecular weight of 40 kDa+/−10%.

In one embodiment a high yield method for manufacture of HEP having a terminal amine is disclosed.

In one embodiment a GSC compound functionalized with a benzaldehyde moiety is provided which is suitable for conjugation with compounds of interest.

In one embodiment a benzaldehyde moiety is attached to the GSC compound, thereby resulting in a GSC-benzaldehyde compound suitable for conjugation to a HEP polymer functionalized with an amine group (cf. FIG. 1).

In one embodiment, 4-formylbenzoic acid is chemically coupled to a HEP polymer and subsequently coupled to GSC by reductive amination (cf. FIG. 2).

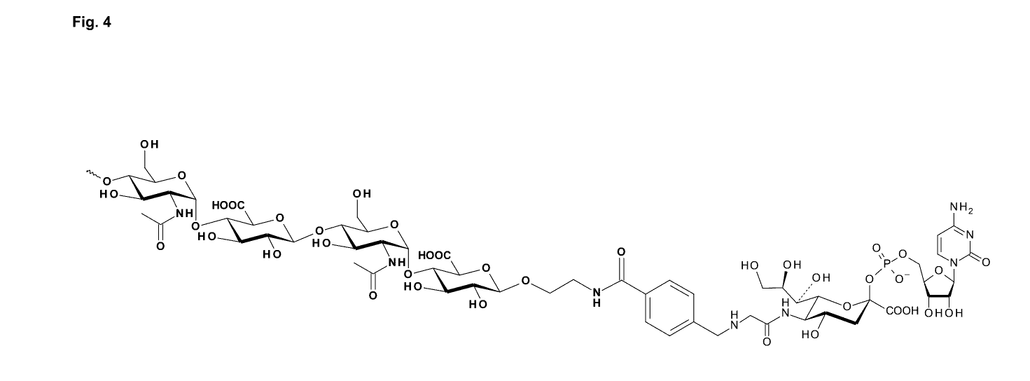

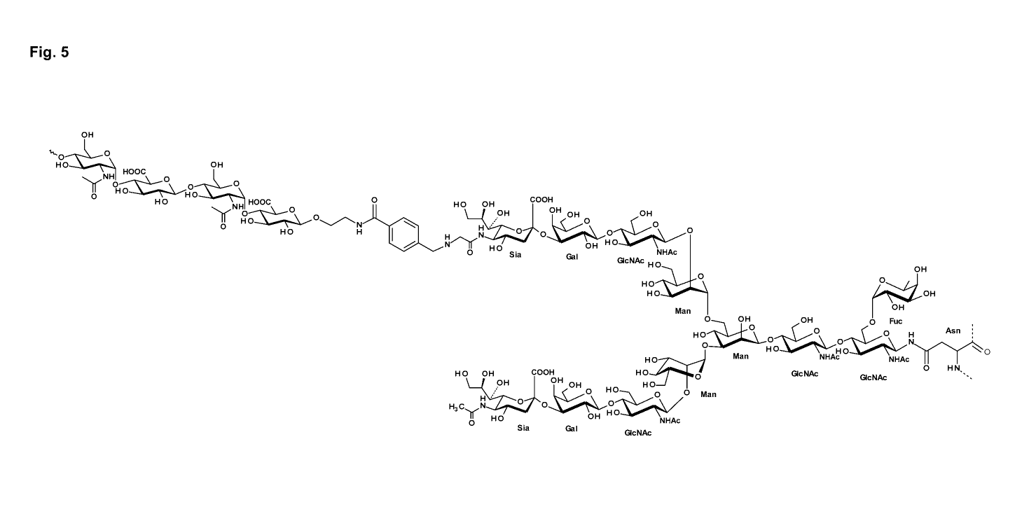

In a preferred embodiment the invention provides a GSC-based reagent wherein a 4-methylbenzoyl sublinker connects HEP and GSC (cf. FIG. 4).

In one embodiment a HEP polymer is conjugated to a FIX polypeptide using 4-methylbenzoyl-GSC based conjugation.

In one embodiment, a HEP polymer moiety comprising an amino group is reacted with 4-formylbenzoic acid and subsequently coupled to the glycyl amino group of GSC by a reductive amination.

In one embodiment a HEP polymer comprising a reactive amine is conjugated to a GSC compound functionalized with a benzaldehyde moiety, wherein said amine is reacted with said benzaldehyde moiety to yield a linker between HEP and GSC which comprises a 4-methylbenzoyl sublinking moiety.

In another embodiment a HEP polymer is functionalized with a reactive benzaldehyde is conjugated to the glycyl amine part of a GSC compound, wherein said benzaldehyde is reacted with an amine to yield a linker between HEP and GSC which comprises a 4-methylbenzoyl sublinking moiety.

In one embodiment the HEP-GSC conjugate is further conjugated onto a FIX polypeptide to yield a conjugate wherein the HEP polymer is linked to said FIX polypeptide via a 4-methylbenzoyl sublinking moiety.

In one embodiment GSC prepared according to WO2007056191 is reacted with a HEP polymer moiety comprising a benzaldehyde moiety under reducing conditions.

In one embodiment various HEP-benzaldehyde compounds suitable for coupling to GSC are provided.

In one embodiment the sublinker between HEP and GSC is not able to form sterio- or regio isomers.

In one embodiment the sublinker between HEP and GSC is not able to form sterio- or regio isomers, and therefore has lesser potential for generating immune response in humans.

In one embodiment the HEP polymer is linked to the FIX polypeptide using a chemical linker comprising 4-methylbenzoyl-GSC.

In one embodiment HEP-GSC is used for preparing a FIX polypeptide N-glycan HEP conjugate (cf. FIG. 5).

In one embodiment HEP-GSC is used for preparing a FIX polypeptide N-glycan HEP conjugate using ST3GaIIII.

In one embodiment HEP-GSC is used for preparing a FIX polypeptide O-glycan HEP conjugate using ST3GaII.

In one embodiment the HEP polymer is linked to an N-glycan on the FIX activation peptide, such as N157 or N167 of SEQ ID NO: 1.

In another embodiment the HEP polymer is linked to an O-glycan on the FIX activation peptide, such as an O-glycan in position 159, 169 or 172 of SEQ ID NO: 1.

In one embodiment the selected HEP polymer size allows for an in vivo useful half-life while at the same time retaining appropriate in vivo activation into FIXa while also having a suitable viscosity in liquid solution.

In one embodiment a HEP polymer size below 73 kDa is selected to arrive at a suitable viscosity in liquid formulation.

In one embodiment a HEP polymer size below 52 kDa is selected to arrive at a suitable viscosity in liquid formulation.

In one embodiment a HEP polymer size of 40 kDa or less is selected to arrive at a suitable viscosity in liquid formulation.

In one embodiment, a CMP activated sialic acid derivative used in the present invention is represented by the following structure:

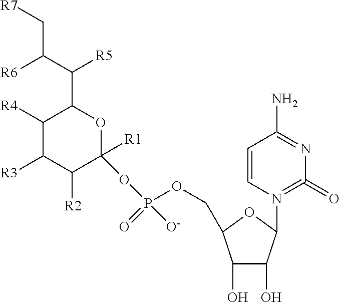

wherein R1 is selected from —COOH, —CONH₂, —COOMe, —COOEt, —COOPr and R2, R3, R4, R5, R6 and R7 independently can be selected from —H, —NH₂, —SH, —N3, —OH, —F or a glycylamido group such as —NHC(O)CH₂NH₂.

In one embodiment, R1 is —COOH, R2 is H, R3=R5=R6=R7=—OH and R4 is —NHC(O)CH₂NH₂ and the sialic acid derivative is CMP activated.

In one embodiment the CMP activated sialic acid is GSC having the following structure:

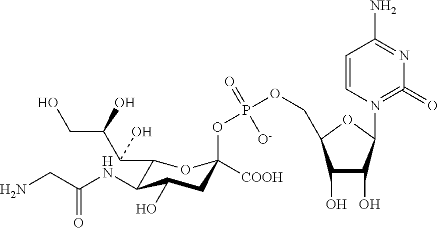

In one embodiment the sialic acid derivative is connected to a FIX polypeptide glycan following removal of the CMP group and has the following structure:

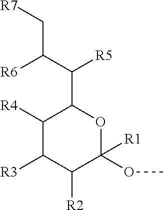

wherein the open valence bond represents the bond to FIX, and

wherein R1 is selected from —COOH, —CONH₂, —COOMe, —COOEt, —COOPr and R2, R3, R4, R5, R6 and R7 can independently be selected from —H, —NH₂, —SH, —N3, —OH, —F or a glycylamido group such as —NHC(O)CH₂NH₂.

In one embodiment a HEP polymer is connected to the glycylamido group of a said sialic acid derivative.

The following is a non-limiting list of aspects of the present invention:
1. A method of linking a half-life extending moiety having a reactive amine to a GSC moiety having a reactive amine, wherein the reactive amine on the half-life extending moiety is first reacted with an activated 4-formylbenzoic acid to yield the compound of Formula A1:

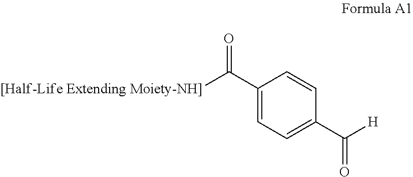

which is subsequently reacted with a GSC moiety under reducing conditions to yield a compound according to Formula A2:

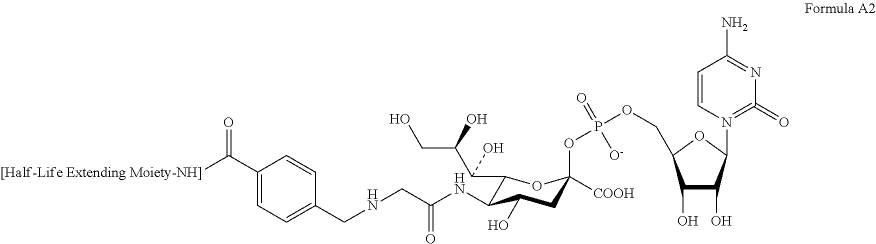

2. A method of linking a half-life extending moiety having a reactive amine to a GSC moiety having a reactive amine, wherein the reactive amine on the GSC moiety first is reacted with an activated 4-formylbenzoic acid to yield a compound according to Formula A3:

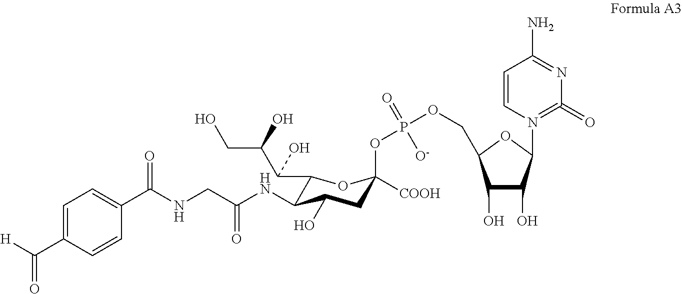

which is subsequently reacted with the reactive amine on the half-life extending moiety under reducing conditions to yield a compound according to Formula A4:

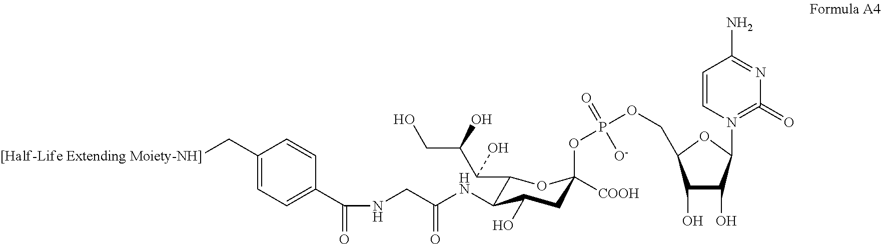

3. The method according to any one of aspects 1 to 3 wherein the half-life extending moiety is a heparosan polymer.
4. A method according to aspect 1 wherein a heparosan polymer modified with a 4-formylbenzoyl group (AA1):

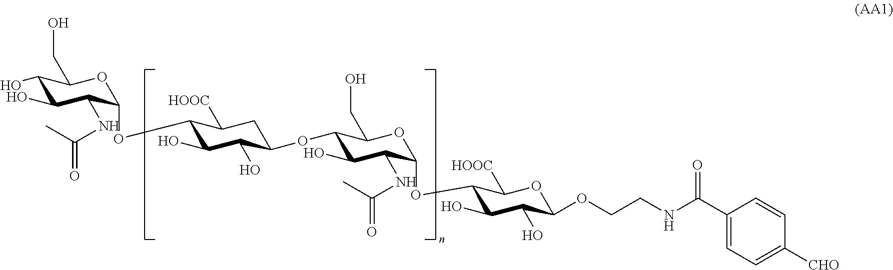

is reacted with GSC (BB1) in the presence of a reducing agent

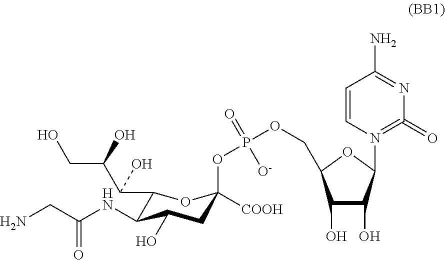

to yield the reagent (CC1):

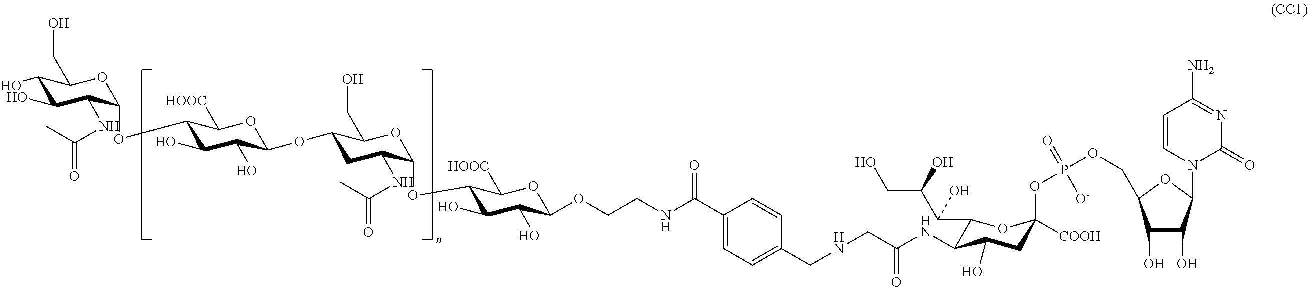

wherein n is an integer from 5 to 450.
5. The method according to any one of aspects 1 to 4 further comprising a subsequent step wherein the half-life extending moiety conjugated to GSC is enzymatically conjugated to a Factor IX polypeptide to yield a conjugate wherein the half-life extending moiety is attached to the protein via a linker comprising a 4-methylbenzoyl sublinker and lacking the cytidine monophosphate group of GSC.
6. A product obtainable by the method according to any one of aspects 1 to 5.
   The invention is further described by the following non-limiting embodiments:
1. A conjugate comprising a Factor IX polypeptide, a linking moiety, and a heparosan polymer wherein the linking moiety connecting the Factor IX polypeptide and the heparosan polymer comprises X as follows:

[heparosan polymer]-[X]-[Factor IX polypeptide]
wherein X comprises a sialic acid derivative which connects a moiety according to Formula E1 below to the Factor IX polypeptide:

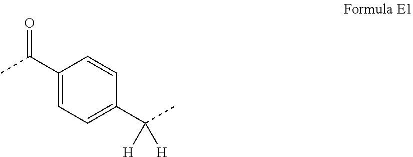

2. The conjugate according to embodiment 1 wherein the sialic acid derivative is a sialic acid derivative according to Formula E2 below:

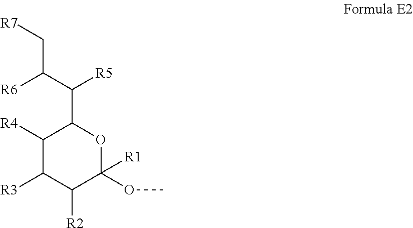

wherein the group in position R1 is selected from the group comprising —COOH, —CONH₂, —COOMe, —COOEt, —COOPr and the group in position R2, R3, R4, R5, R6 and R7 can independently be selected from a group comprising —H, —NH—, —NH₂, —SH, —N3, —OH, —F or —NHC(O)CH₂NH—.
3. The conjugate according to embodiment 2 wherein the sialic acid derivative is a glycyl sialic acid according to Formula E3 below:

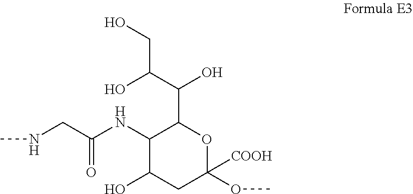

and wherein the moiety of Formula 1 is connected to the terminal —NH handle of Formula E3.
4. The conjugate according to embodiment 1, 2 or 3 wherein

[heparosan polymer]-[X]-

comprises the structural fragment shown in Formula E4 below:

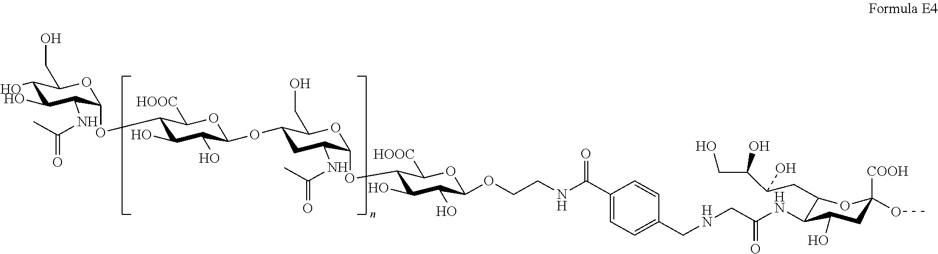

wherein n is an integer from 5 to 450.
5. A conjugate comprising a Factor IX polypeptide and a heparosan polymer wherein said heparosan polymer has a molecular weight in the range 5 to 100 kDa.
6. The conjugate according to embodiment 5 wherein the heparosan polymer has a molecular weight in the range 13 to 60 kDa.
7. The conjugate according to embodiment 5 wherein the heparosan polymer has a molecular weight in the range 27 to 40 kDa.
8. The conjugate according to embodiment 5 wherein the molecular weight of the heparosan polymer is 40 kDa+/−10%.
9. A pharmaceutical composition comprising the conjugate according to any one of embodiments 1 to 8.
10. Use of a heparosan polymer conjugated to a Factor IX polypeptide in aPTT assays wherein the variability in recovery of Factor IX activity is less than 523 percentage points.
11. Use of a heparosan polymer conjugated to a Factor IX polypeptide according to embodiment 10 wherein the variability in recovery of Factor IX activity is no more than 115 percentage points.
12. The conjugate according to any one of embodiments 1 to 8 for use as a medicament.
13. The conjugate according to any one of embodiments 1 to 8 for use in the treatment of coagulopathy.
14. The conjugate according to any one of embodiments 1 to 8 for use in the treatment of haemophilia B.
15. The conjugate according to any one of embodiments 1 to 8 for use in prophylactic treatment of haemophilia B.
16. A method of conjugating a heparosan polymer to a Factor IX polypeptide comprising the steps of:
   a) reacting a heparosan polymer comprising a reactive amine [HEP-NH] with an activated 4-formylbenzoic acid to yield the compound of Formula E5 below,

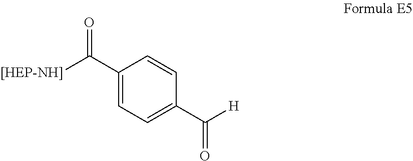

wherein [HEP-NH] represents any HEP polymer functionalized with a terminal primary amine,
b) reacting the compound of Formula 5 with a CMP-activated sialic acid derivative under reducing conditions
c) conjugating the compound obtained in step b) to a glycan on the Factor IX polypeptide.
17. The method according to embodiment 16 wherein the CMP activated sialic acid derivative used in step b) has the following Formula E6:

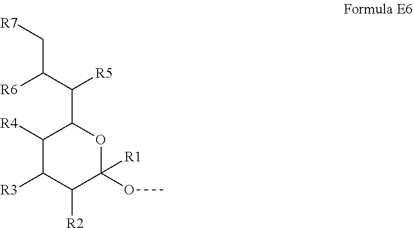

wherein the group in position R1 is selected from the group comprising —COOH, —CONH2, —COOMe, —COOEt, —COOPr and the group in position R2, R3, R4, R5, R6 and R7 can independently be selected from a group comprising —H, —NH—, —NH₂, —SH, —N3, —OH, —F or NHCOCNH₂.
18. The method according to embodiment 16 or 17 wherein R4 is NHCOCNH₂.
19. Conjugates obtainable using the method according to embodiment 16, 17 or 18.

The present invention is further illustrated by the following examples which, however, are not to be construed as limiting the scope of protection. The features disclosed in the foregoing description and in the following examples may, both separately and in any combination thereof, be material for realising the invention in diverse forms thereof.

### EXAMPLES

### Abbreviations Used in Examples

AUS: Arthrobacter ureafaciens sialidase
CMP: Cytidine monophosphate
EDTA: Ethylenediaminetetraacetic acid
Gla: Gamma-carboxyglutamic acid
GlcUA: Glucuronic acid
GlcNAc: N-acetylglucosamine
Grx2: Glutaredoxin II
GSC: Glycyl sialic acid cytidine monophosphate
GSC-SH: [(4-mercaptobutanoyl)glycyl]sialic acid cytidine monophosphate
GSH: Glutathione
GSSG: Glutathione disulfide
HEP: HEParosan
HEP-FIX: Heparosan conjugated to Factor IX polypeptide (used interchangeably with FIX-HEP)
HEP-[C]-FIX(E162C): HEParosan conjugated via cysteine to FIX(E162C).
HEP-[N]-FIX: HEParosan conjugated via N-glycan to FIX.
HEP-GSC: GSC-functionalized heparosan polymers
HEP-NH₂: Amine functionalized HEParosan polymer
HEPES: 2-[4-(2-hydroxyethyl)piperazin-1-yl]ethanesulfonic acid
His: Histidine
IV: Intravenous
KO: Knock-out
MRT: Mean Residence Time
PABA: p-aminobenzamidine
PmHS1: Pasteurella mutocida Heparosan Synthase I
pNA: para-nitroaniline
SXa-11: Factor Xa chromogenic substrate
UDP: Uridine diphosphate

### Example 1

### Quantification Method

The conjugates of the invention were analysed for purity by HPLC. HPLC was also used for conjugate quantifications. Quantifications were based on area under curve integration using 280 nm wavelength absorption profile. BeneFIX® recombinant coagulation Factor IX manufactured by Wyeth Pharmaceuticals Inc. was used as reference. A Zorbax 300SB-C3 column (4.6×50 mm; 3.5 μm Agilent, Cat. No.: 865973-909) was used. The column was operated on an Agilent 1100 Series HPLC furnished with fluorescence detector (Ex 280 nm, Em 348 nm). Column temperature was 30° C., with 5 μg sample injection and a flow rate of 1.5 ml/min. Column was eluted with a water (A)-acetonitrile (B) solvent system containing 0.1% trifluoroacetic acid. The gradient program was as follows: 0 min (25% B); 4 min (25% B); 14 min (46% B); 35 min (52% B); 40 min (90% B); 40.1 min (25% B).

### Example 2

### SDS-PAGE Analysis

SDS PAGE analysis was performed using precast Nupage 7% tris-acetate gel, NuPage tris-acetate SDS running buffer and NuPage LDS sample buffer all from Invitrogen. Samples were denaturized (70° C. for 10 min.) before analysis. HiMark HMW (Invitrogen) was used as standard. Electrophoresis was run in XCell Surelock Complete with power station (Invitrogen) for 80 min at 150 V, 120 mA. Gels were stained using SimplyBlue SafeStain from Invitrogen.

### Example 3

### Selective Reduction of FIX(E162C)

FIX(E162C) was reduced using a glutathione based redox buffer system, in similar manner as described for FVIIa407C in US20090041744. Non-reduced FIX(E162C) (10.5 mg) was incubated for 23 hours at room temperature in a total volume of 5.25 ml 50 mM Hepes, 100 mM NaCl, 10 mM CaCl₂, pH 7.0 containing 0.5 mM GSH, 15 μM GSSG, 2.5 mM p-aminobenzamidine and 2 μM Grx2. The reaction mixture was subsequently diluted to 44 ml with 50 mM Hepes, 100 mM NaCl, cooled on ice and added to 4 ml 100 mM EDTA solution while keeping pH at 7.0. The entire content was then loaded onto 2×5 ml HiTrap Q FF column (Amersham Biosciences, GE Healthcare) equilibrated in buffer A (50 mM Hepes, 100 mM NaCl, pH 7.0) to capture FIX(E162C). After wash with buffer A to remove unbound Grx2, FIX (E162C) was eluted in one step with buffer B (50 mM Hepes, 1 M NaCl, 10 mM CaCl₂, pH 7.0). The concentration of FIX(E162C) in the eluate was determined by HPLC. p-aminobenzamidine (20 μl of an aqueous 0.5M solution) was then added to a final concentration of 2 mM. 7.95 mg of single cysteine reduced FIX(E162C) was isolated in 5 ml of 50 mM Hepes, 1 M NaCl, 10 mM CaCl₂, 2 mM p-aminobenzamidine, pH 7.0.

### Example 4

### Synthesis of 60 kDa HEP-[C]-FIX(E162C)

A solution of single cysteine reduced FIX(E162C) (7.95 mg) in 50 mM Hepes, 1 M NaCl, 10 mM CaCl₂, 2 mM PABA, pH 7.0 (5 ml) was added 60 kDa HEP-maleimide (55.3 mg) dissolved in 50 mM Hepes, 100 mM NaCl, 10 mM CaCl₂, pH 7.0 (2.95 ml). The clear solution was placed on a roller mixer, and gently rotated for 22 hours at room temperature. The reaction mixture was then loaded onto a FIX specific affinity column (CV=66 ml with total binding capacity of 13.3 mg FIX) modified with a Gla-domain specific antibody and step eluted first with 2 column volumes of buffer A (50 mM Hepes, 100 mM NaCl, 10 mM CaCl₂, pH 7.4) then two column volumes of buffer B (50 mM Hepes, 100 mM NaCl, 10 mM EDTA, pH 7.4). Fractions containing FIX and 60 kDa HEP-FIX conjugate were collected and loaded directly onto a 2×5 ml HiTrap Q FF ion-exchange column (Amersham Biosciences, GE Healthcare) that was pre-equilibrated with 10 mM His, 100 mM NaCl, pH 7.5. The column was washed with 4 column volumes of 10 mM His, 100 mM NaCl, pH 7.5 to remove un-bound material. The eluent was then changed to buffer A (10 mM His, 100 mM NaCl, 10 mM CaCl₂, pH=6.0). Un-modified FIX(E162C) was eluted with 5 column volumes of 20% buffer B (10 mM His, 100 mM NaCl, 10 mM CaCl₂, pH=6.0), and 60 kDa HEP-FIX subsequently with 5 column volumes of 40% buffer B. The fractions containing conjugate were combined, and dialyzed against 10 mM His, 100 mM NaCl, 10 mM CaCl₂, pH=6.0 using a Slide-A-Lyzer cassette (Thermo Scientific) with a cut-off of 10 kDa. The final volume was adjusted to 0.3 mg/ml by addition of 10 mM His, 100 mM NaCl, 10 mM CaCl₂, pH=6.0. Conjugate was analysed for purity on SDS-PAGE as described in example 2. Yield of conjugate was 3.75 mg (47%) as determined by HPLC quantification against FIX standard.

### Example 5

### Synthesis of 38.8 kDa HEP-[C]-FIX(E162C)

This conjugate was prepared as described in example 4 using single cysteine reduced FIX(E162C) (6.30 mg) prepared as described in example 1 and 38.8 kDa HEP-maleimide (18.9 mg). 2.8 mg (44%) 38.8 kDa HEP-[C]-FIX(E162C) was isolated in 6.3 ml 10 mM His, 150 mM NaCl, 5 mM CaCl₂, 0.005% Tween80, pH 6.4 (8 μM; 0.45 mg/ml).

### Example 6

### Synthesis of 27 kDa HEP-[C]-FIX(E162C)

This conjugate was prepared as described in example 4 using single cysteine reduced FIX(E162C) (6.32 mg) prepared as described in example 1 and 27 kDa HEP-maleimide (10.2 mg). 3.96 mg (62%) 27 kDa HEP-[C]-FIX(E162C) was isolated in 8.84 ml 10 mM His, 150 mM NaCl, 5 mM CaCl₂, 0.005% Tween80, pH 6.4 (8 μM; 0.45 mg/ml).

### Example 7

### Synthesis of 13 kDa HEP-[C]-FIX(E162C)

This conjugate was prepared analogous to example 4 using single cysteine reduced FIX(E162C) (10.0 mg) prepared as described in example 1 and 13 kDa HEP-maleimide (10.0 mg). 2.3 mg (23%) 13 kDa HEP-[C]-FIX(E162C) was isolated in 5.10 ml 10 mM His, 150 mM NaCl, 5 mM CaCl₂, 0.005% Tween80, pH 6.4 (8 μM; 0.45 mg/ml).

### Example 8

### Desialylation of FIX

FIX (20.4 mg) was reacted with sialidase (Arthrobacter ureafaciens, 140 μl, 0.3 mg/ml, 200 U/ml) in 1.7 ml of 10 mM Histidine, 3 mM CaCl₂, 150 mM NaCl pH 6.2, for 1 hour at room temperature. The reaction mixture was then diluted with 50 mM Hepes, 100 mM NaCl, pH 7.0 (20 ml), and cooled on ice. 100 mM EDTA solution (3 ml) was added in small portions. After each addition pH was measured. pH was maintained within 5.5-9.0. The reaction mixture was diluted to 40 ml with MilliQ water to lower the conductivity and applied to a 2×5 ml HiTrap Q FF ion-exchange columns (Amersham Biosciences, GE Healthcare) equilibrated with buffer A (50 mM Hepes, 100 mM NaCl, pH 7.0). AsialoFIX was eluted in one step with buffer B (50 mM Hepes, 1 M NaCl, 10 mM CaCl₂, pH 7.0). The concentration of asialoFIX in the eluate was determined by HPLC. 17.2 mg asialoFIX was isolated in 6 ml 50 mM Hepes, 1 M NaCl, 10 mM CaCl₂, pH 7.0 (2.86 mg/ml).

### Example 9

### Synthesis of [(4-mercaptobutanoyl)glycyl]sialic acid cytidine monophosphate (GSC-SH)

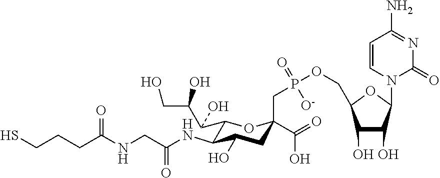

Glycyl sialic acid cytidine monophosphate (200 mg; 0.318 mmol) was dissolved in water (2 ml), and thiobutyrolactone (325 mg; 3.18 mmol) was added. The two phase solution was gently mixed for 21 h at room temperature. The reaction mixture was then diluted with water (10 ml) and applied to a reverse phase HPLC column (C18, 50 mm×200 mm). Column was eluted at a flow rate of 50 ml/min with a gradient system of water (A), acetonitrile (B) and 250 mM ammonium hydrogen carbonate (C) as follows: 0 min (A: 90%, B: 0%, C: 10%); 12 min (A: 90%, B: 0%, C: 10%); 48 min (A: 70%, B: 20%, C: 10%). Fractions (20 ml size) were collected and analysed by LC-MS. Pure fractions were pooled, and passed slowly through a short pad of Dowex 50 W×2 (100-200 mesh) resin in sodium form, before lyophilized into dry powder. Content of title material in freeze dried powder was then determined by HPLC using absorbance at 260 nm, and glycyl sialic acid cytidine monophosphate as reference material. For the HPLC analysis, a Waters X-Bridge phenyl column (5 μm 4.6 mm×250 mm) and a water acetonitrile system (linear gradient from 0-85% acetonitrile over 30 min containing 0.1% phosphoric acid) was used. Yield: 61.6 mg (26%). LCMS: 732.18 (MH⁺); 427.14 (MH⁺-CMP). Compound was stable for extended periods (>12 months) when stored at −80° C.

### Example 10

### Synthesis of Heparosan Polymer with Terminal Amino Ethyl Handle

### Step 1: Synthesis of (2-Fmoc-amino)ethyl 2,3,4-tri-O-acetyl-β-D-glucuronic acid methyl ester

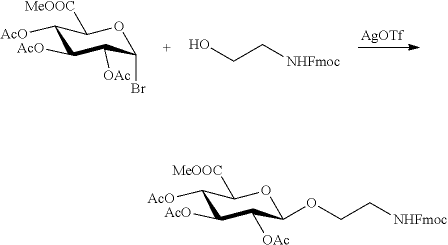

Powdered molecular sieves (1.18 g, 4 Å) were heated at 110° C. in a 50 ml round bottom flask fitted with a magnetic stir bar overnight, flushed with argon, and allowed to cool to room temperature. 900 mg (2.19 mmol) aceto-bromo-β-D-glucuronic acid methyl ester and 748.5 mg (2.64 mmol, 1.2 eq) 2-(Fmoc-amino)ethanol were added under argon, followed by 28 ml dichloromethane. The suspension was stirred for 15 minutes at room temperature and then cooled on an ice/NaCl-slurry for 30 minutes. A white precipitate formed during the cooling process. 676.3 mg (2.63 mmol, 1.2 eq) silver trifluoromethanesulfonate (AgOTf) was added in 3 portions over a period of ˜5 minutes. After 20 minutes the ice-bath was removed. The previously noted white precipitate started dissolving, while at the same time a grey precipitate started to form. The reaction was stirred overnight at room temperature and then quenched by addition of 190 μL triethylamine (2.63 mmol, 1.2 eq). After filtration through a thin Celite 521 pad (˜0.1-0.2 cm deep), and subsequent washing of the filter cake with 20 ml dichloromethane, the combined filtrates were diluted with dichloromethane to 150 ml. The organic phase was washed with 5% NaHCO₃ (1×50 mL) and water (1×50 mL), then dried over magnesium sulfate and filtered. The filtrate was concentrated in vacuo on a rotary evaporator (≦40° C. water bath) to dryness and then re-dissolved in 2 mL dichloromethane. The solution was injected onto a VersaPak silica gel flash column (23×110 mm, 23 g) and the product eluted with 50% ethyl acetate in hexanes. The product-containing fractions were identified by TLC (ethyl acetate:hexanes, 1:1), and concentrated in vacuo on a rotary evaporator (≦40° C. water bath) to dryness. Trituration of the obtained residue with ˜10 mL diethyl ether yielded the title material as a white crystalline foam. Yield: 293 mg (0.49 mmol, 22.4%).

### Step 2: Synthesis of (2-Fmoc-amino)ethyl β-D-glucuronic acid, sodium salt

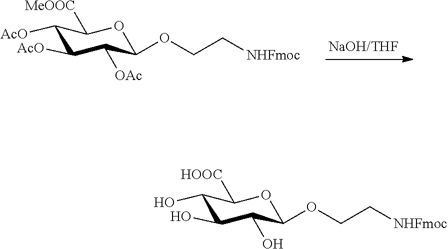

490 mg (0.817 mmol, 1 eq) of (2-Fmoc-amino)ethyl 2,3,4-tri-O-acetyl-β-D-glucuronic acid methyl ester obtained in step 1 was dissolved in 47.5 mL methanol and 2.5 mL (2.45 mmol, 3 eq) of a 1 M NaOH-solution was slowly added under stirring. The reaction was monitored by TLC using 1-butanol:acetic acid:water=1:1:1 as eluent. After TLC showed complete consumption of the methyl ester, the pH of the reaction mixture was lowered to pH 8-9 by addition of 1 N HCl. 204 mg (2.45 mmol, 3 eq) solid NaHCO₃ followed by 241.7 mg (0.899 mmol, 1.1 eq) Fmoc-chloride was then added. When TLC analysis showed completion of reaction, the reaction mixture was diluted with ˜150 mL water, extracted twice with ethyl acetate (2×30 mL), and then concentrated in vacuo over a 40° C. water bath to about 20 mL to remove any remaining organic solvents. The solution was acidified by addition of acetic acid to a content of ˜5% (v:v), and passed through a 5 gram Strata C-18E SPE tube (pre-wetted in methanol, and equilibrated in 5% acetic acid according to manufacturer's instructions). The resin was washed with 5% acetic acid, and the product was eluted with a mixture of 90% methanol with 10% Tris.HCl, pH 7.2 (v:v). After concentration in vacuo (<40° C. water bath) to dryness, the residue was redissolved and the pH was adjusted to pH 7.2 with sodium hydroxide. This solution was used directly as stock solution in the synthesis of (2-Fmoc-amino)ethyl 4-O-(2-deoxy-2-acetamido-α-D-glucopyranosyl)-β-D-glucuronic acid below without further purification.

### Step 3: Synthesis of (2-Fmoc-amino)ethyl 4-O-(2-deoxy-2-acetamido-α-D-glucopyranosyl)-β-D-glucuronic acid, sodium salt

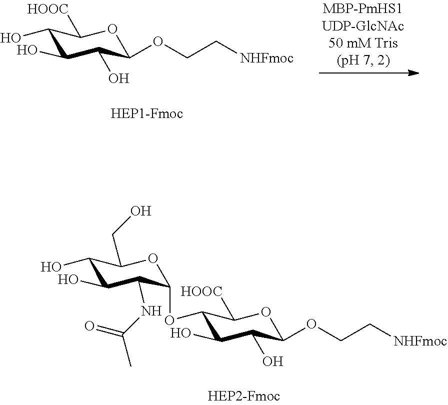

To a solution of 380 mg (2-Fmoc-amino)ethyl β-D-glucuronic acid obtained in step 2 (0.83 mmole, 1 eq) in 100.8 mL water was added 5.6 mL 1 M Tris.HCl, pH 7.2, 5.6 mL 100 mM MnCl₂, and 1.8 g UDP-GlcNAc (2.79 mmole, 3.4 eq). After slow addition of 5.1 mL MBP-PmHS1 enzyme (15.47 mg/mL; 78.9 mg) over ˜1 min, the reaction was left to stir slowly at room temperature until TLC analysis (1-butanol:acetic acid:water=2:1:1) showed nearly complete conversion of starting material. The solution was acidified by addition of 2.8 mL acetic acid to precipitate the spent MBP-PmHS1 and transferred into 50 mL centrifuge bottles. The solution was then centrifuged for 30 min at 10,000 rpm in a JM-12 rotor (˜16,000×g) at room temperature. The supernatant was decanted and added 160 mL methanol. The pellet was extracted 4×25 mL with a solution of water:methanol:acetic acid=45:50:5 (v:v:v). The combined supernatant and extracts were passed through 2 g Strata-SAX tubes (equilibrated in water:methanol:acetic acid=45:50:5 (v:v:v)) to remove any UDP & UDP-GlcNAc (complete removal required 28 grams of resin). The target molecule was unretained and passed through the resin under these conditions; while the more highly charged UDP & UDP-GlcNAc were retained. The combined eluates were concentrated in vacuo (water batch; ≦40° C.), re-dissolved in water, and the pH was adjusted to pH 7.2 using sodium hydroxide. This solution was used directly in the next step without further purification.

### Step 4: Synthesis of (2-Fmoc-amino)ethyl 4-O-(2-deoxy-2-acetamido-4-O-(β-D-glucopyranosyluronic acid)-α-D-glucopyranosyl)-β-D-glucuronic acid, disodium salt

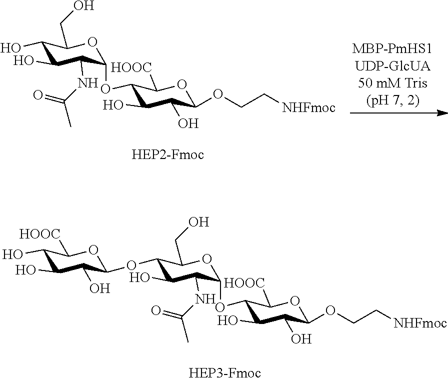

An aqueous solution (38 ml) containing 9 mM (2-Fmoc-amino)ethyl 4-O-(2-deoxy-2-acetamido-α-D-glucopyranosyl)-β-D-glucuronic acid, 30 mM UDP-GlcUA, 50 mM Tris.HCl, and 5 mM MnCl₂ was placed in a spinner flask. Over a period ˜1 min, 9.5 mL MBP-PmHS1 was added dropwise under slow agitation. The reaction mixture was left to stir overnight, after which TLC analysis (eluent: n-BuOH:AcOH:H2O=4:1:1 (v:v:v)) showed complete conversion of the starting material. The reaction mixture was filtered through a 1 μm glass fiber syringe filter, and passed through a 5 gram C18-E SPE tube (equilibrated in water, following manufacturer's instructions). The resin was washed with water, followed by elution of the target molecule with a mixture of 90% aqueous MeOH, 1 mM Tris.HCl, pH 7.2. The eluate was concentrated in vacuo (waterbath ≦40° C.), then re-dissolved in 25 mL 10 mM Tris.HCl, pH 7.2, and filtered through a 0.2 μm SFCA syringe filter. The filtrate containing the target molecule was further purified by anion exchange chromatography. An Akta Explorer 100 furnished with a 2.6×13 cm Q Sepharose HP column and operated with Unicorn 5.11 software was used. Two buffer systems (buffer A: 10 mM Tris.HCl, pH 7.2 and buffer B: 10 mM Tris.HCl, pH 7.2, 1 M NaCl) were used for elution. The target molecule was eluted using a 0-20% B gradient over 175 min; at a flowrate of 10 ml/min. 10 ml fraction were collected. The fractions containing product were combined, concentrated on a rotary evaporator in vacuo (waterbath <40° C.) to dryness, and used in the next step without further purification.

### Step 5: Synthesis (2-aminoethyl) 4-O-(2-deoxy-2-acetamido-4-O-(β-D-glucopyranosyluronic acid)-α-D-glucopyranosyl)-β-D-glucuronic acid, disodium salt

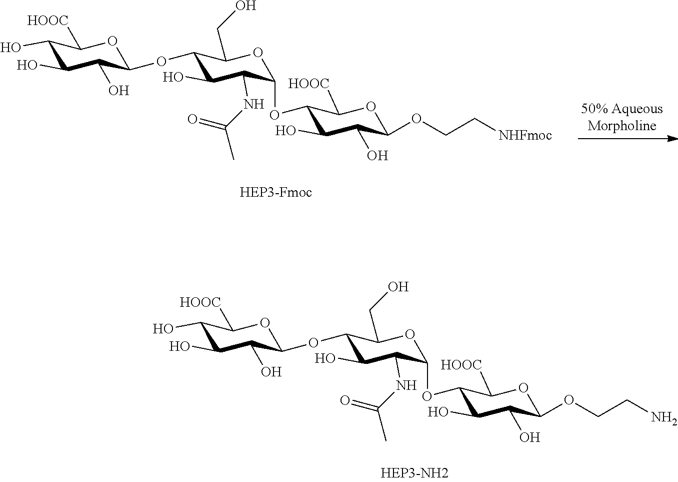

(2-Fmoc-amino)ethyl 4-O-(2-deoxy-2-acetamido-4-O-(β-D-glucopyranosyluronic acid)-α-D-glucopyranosyl)-β-D-glucuronic acid, disodium salt obtained as described in step 4, was dissolved in 4 mL water and cooled on an ice-bath. A volume of 4 mL neat morpholine was added under stirring and the ice bath was removed. Stirring was continued at room temperature, until TLC analysis (n-BuOH:AcOH:H₂O=3:1:1 (v:v:v)) using UV 254 nm detection showed complete consumption of starting material. Reaction was complete within less than 1.5 hrs. The reaction mixture was diluted with ˜50 mL water and extracted three times with 50 mL EtOAc. The aqueous phase containing the target molecule was concentrated on a rotary evaporator in vacuo (waterbath <40° C.) and co-evaporated three times with water. The residue was re-dissolved in 10 mL water and passed through a 1 gram SDB-L SPE column preequilibrated in water. The target passed through the column unretained. The column was washed with 10 mL water and the combined fractions with target were concentrated in vacuo to dryness (water bath; <40° C.). The obtained residue was dissolved in 1.5 mL 1 M NaOAc, pH 7.5, filtered through a 0.2 μm spinfilter, and desalted by size-exclusion chromatography over a Sephadex G-10 column (2×75 cm, 235 mL) with water as eluent. Structure of the title material was confirmed by MALDI-TOF MS (matrix: 5 mg/mL ATT; 50% acetonitrile/0.05% trifluoroacetic acid): 636.83 [M+Na⁺]. After lyophilization, the title material was dissolved in water, the pH of the obtained solution was adjusted to pH 7.0-7.5 by addition of sodium hydroxide, and the trisaccharide content was determined by carbazole assay (Bitter T, Muir H M. Anal Biochem 1962 October; 4:330-4). The obtained stock solution was aliquoted and stored at −80° C. in tightly sealed containers until needed.

The overall isolated yield of (2-aminoethyl) 4-O-(2-deoxy-2-acetamido-4-O-(β-D-glucopyranosyluronic acid)-α-D-glucopyranosyl)-β-D-glucuronic acid starting from (2-Fmoc-amino)ethyl β-D-glucuronic acid was 210 mg (0.34 mmole, 41%).

### Step 6: Production of Heparosan Polysaccharide with Amine Terminal

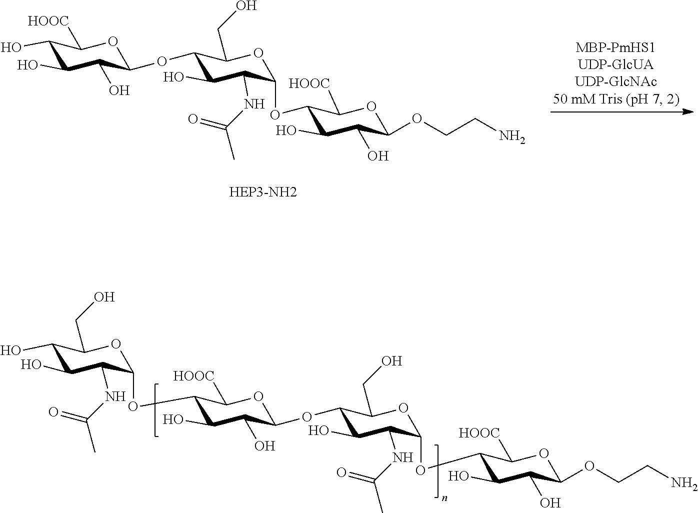

To obtain a heparosan polymer derivative with a free amine group (HEP-NH₂), the Pasteurella multocida heparosan synthase 1 (PmHS1; DeAngelis & White, 2002 J Biol Chem) was used to chemoenzymatically synthesize polymer chains in a parallel fashion in vitro (Sismey-Ragatz et al., 2007 J Biol Chem and U.S. Pat. No. 8,088,604). A fusion of the E. coli maltose-binding protein with PmHS1 was used as the catalyst for elongating the (2-aminoethyl) 4-O-(2-deoxy-2-acetamido-4-O-(β-D-glucopyranosyluronic acid)-α-D-glucopyranosyl)-β-D-glucuronic acid (HEP3-NH₂) obtained in step 5 into longer polymer chains using UDP-GlcNAc and UDP-GlcUA precursors and MnCl₂ catalysis as described in US2010036001.

### Step 7: Production of Heparosan Polysaccharide with Terminal Benzaldehyde Functionality

To obtain a heparosan polymer derivative for coupling via reductive amination, etc. to accessible amino functionalities on the target drug compound, heparosan-NH₂, was coupled with N-succinimidyl-4-formylbenzoic acid, to form a benzaldehyde-modified heparosan polymer. Basically, in one example, N-succinimidyl-4-formylbenzoic acid (Chem-Impex, Inc) dissolved in dimethyl sulfoxide (11.94 mg in 205 mL) was slowly added to a stirred solution of 62.7 g of 43.8 kDa heparosan polymer-NH₂ dissolved in 380 mL 1M sodium phosphate, pH 7.0, 2180 ml water, and 1040 mL dimethylsulfoxide. The reaction mixture was left to stir at room temperature overnight, followed by alcohol precipitation at ambient temperature. The pellet with product was dissolved in 3 L of 500 mM sodium acetate, pH 6.8, further purified and then concentrated by cross flow filtration.

### Example 11

### Synthesis of HEP-Maleimide and HEP-Benzaldehyde Polymers

Maleimide and aldehyde functionalized HEP polymers of defined size are prepared by an enzymatic (PmHS1) polymerization reaction using the two sugar nucleotides UDP-GlcNAc and UDP-GlcUA. A priming trisaccharide (GlcUA-GlcNAc-GlcUA)NH₂ is used for initiating the reaction, and polymerization is run until depletion of sugar nucleotide building blocks. The terminal amine (originating from the primer) is then functionalized with suitable reactive groups, in this case either a maleimide functionality designed for conjugation to free cysteines and thioGSC derivatives, or a benzaldehyde functionality designed for reductive amination chemistry to GSC. Size of HEP polymers can be pre-determined by variation in sugar nucleotide: primer stoichiometry. The technique is described in detail in US2010/0036001.

HEP-benzaldehydes can be prepared by reacting amine functionalized HEP polymers with a surplus of N-succinimidyl-4-formylbenzoic acid (Nano Letters (2007) 7(8), pp. 2207-2210) in aqueous neutral solution. The benzaldehyde functionalized polymers may be isolated by ion-exchange chromatography, size exclusion chromatography, or HPLC.

HEP-maleimides can be prepared by reacting amine functionalized HEP polymers with a surplus of N-maleimidobutyryl-oxysuccinimide ester (GMBS; Fujiwara, K., et al. (1988) J Immunol Meth 112, 77-83).

The benzaldehyde or maleimide functionalized polymers may be isolated by ion-exchange chromatography, size exclusion chromatography, or HPLC. Any HEP polymer functionalized with a terminal primary amine (HEP-NH₂) may be used in the present examples. Two options are shown below:

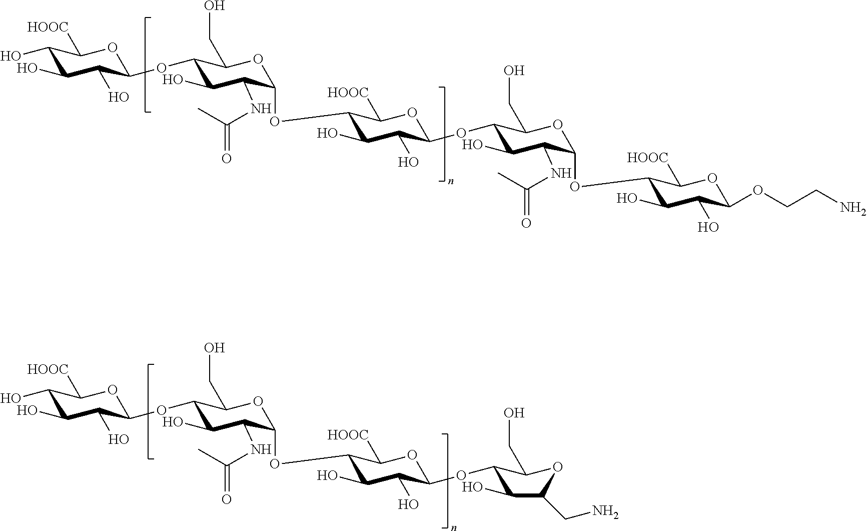

Furthermore the terminal sugar residue in the non-reducing end of the polysaccharide can be either N-acetylglucosamine or glucuronic acid (glucuronic acid is drawn above). Typically a mixture of both is to be expected if equimolar amount of UDP-GlcNAc and UDP-GlcUA has been used in the polymerization reaction.

### Example 12

### Synthesis of 38.8 kDa HEP-GSC Reagent with Succinimide Sublinkage

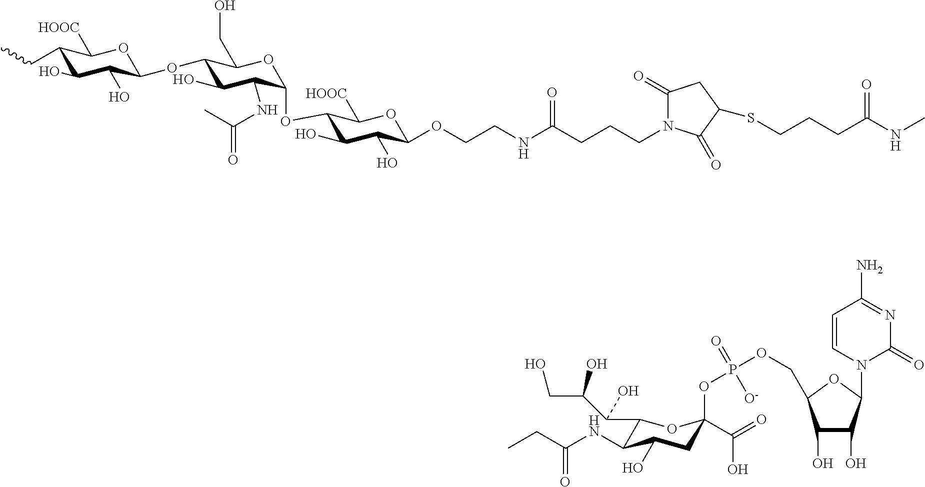

### Example 12

### Continued

The HEP reagent was prepared by coupling GSC-SH ([(4-mercaptobutanoyl)glycyl]sialic acid cytidine monophosphate) with HEP-maleimide in a 1:1 molar ratio as follows: to GSC-SH (0.50 mg) dissolved in 50 mM Hepes, 100 mM NaCl, pH 7.0 (50 μl) was added 26.38 mg of the 38.8 kDa HEP-maleimide dissolved in 50 mM Hepes, 100 mM NaCl, pH 7.0 (1350 μl). The clear solution was left for 2 hours at 25° C. The excess of GSC-SH was removed by dialysis, using a Slide-A-Lyzer cassette (Thermo Scientific) with a cut-off of 10 kDa. The dialysis buffer was 50 mM Hepes, 100 mM NaCl, 10 mM CaCl₂, pH 7.0. The reaction mixture was dialyzed twice for 2.5 hours. The recovered material was used as such in example 14, assuming a quantitative reaction between GSC-SH and HEP-maleimide. The HEP-GSC reagent made by this procedure will contain a HEP polymer attached to sialic acid cytidine monophosphate via a succinimide linkage.

### Example 13

### Synthesis of 60 kDa HEP-GSC with Succinimide Sublinkage

This molecule was prepared using 60 kDa HEP-maleimide and [(4-mercaptobutanoyl)glycyl]sialic acid cytidine monophosphate in a similar way as described for 38.8 kDa HEP-GSC above.

### Example 14

### Synthesis of 38.8 kDa HEP-[N]-FIX with Succinimide Sublinkage

38.8 kDa HEP-[N]-FIX was synthesized as follows. To asialoFIX (17.2 mg) in 50 mM Hepes, 1 M NaCl, 10 mM CaCl₂, pH 7.0 (6 ml) was added 38.8 kDa HEP-GSC (26.38 mg from example 11) in 50 mM Hepes, 100 mM NaCl, 10 mM CaCl₂, pH 7.0 (1.5 ml), followed by rat ST3GaIIII enzyme (3.3 mg; 1.1 unit/mg) in 20 mM Hepes, 120 mM NaCl, 50% glycerol, pH 7.0 (6 ml). The reaction mixture was incubated for 17.5 hours at 32° C. A solution of 157 mM N-acetylneuraminic acid cytidine monophosphate in 50 mM Hepes, 150 mM NaCl, 10 mM CaCl₂, pH 7.0 (0.2 ml) was then added, and the reaction was incubated at 32° C. for an additional hour. The 38.8 kDa HEP-[N]-FIX was then isolated by a combination of affinity and anion-exchange chromatography essentially as described in example 4. The isolated compound was dialyzed into 10 mM His, 150 mM NaCl, 5 mM CaCl₂, 0.005% Tween80, pH 6.4. Conjugate was analyzed for purity on SDS-PAGE as described in example 2. 2.76 mg (16%) of 38.8 kDa HEP-[N]-FIX was isolated in 6.2 ml 10 mM His, 150 mM NaCl, 5 mM CaCl₂, 0.005% Tween80, pH 6.4 (0.45 mg/ml).

### Example 15

### Synthesis of 60 kDa HEP-[N]-FIX with Succinimide Sublinkage

This conjugate was prepared analogous to example 14 using asialoFIX (8.5 mg) prepared as described in example 8 and 60 kDa HEP-GSC (30.0 mg) as prepared in example 13. 0.69 mg (8%) 60 kDa HEP-[N]-FIX was isolated in 1.5 ml 10 mM His, 150 mM NaCl, 5 mM CaCl₂, 0.005% Tween80, pH 6.4 (0.45 mg/ml).

### Example 16

### Synthesis of 41.5 kDa HEP-GSC Reagent with 4-Methylbenzoyl Sublinkage

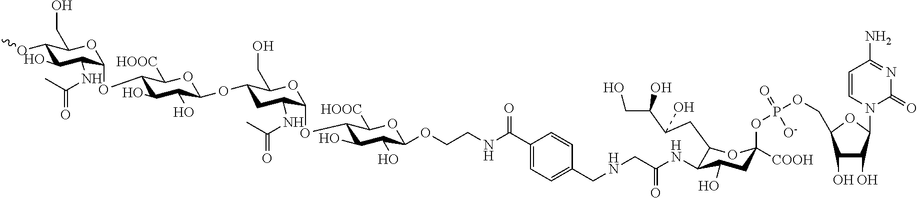

### Example 16

### Continued

Glycyl sialic acid cytidine monophosphate (GSC) (20 mg; 32 μmol) in 5.0 ml 50 mM Hepes, 100 mM NaCl, 10 mM CaCl₂ buffer, pH 7.0 was added directly to dry 41.5 kDa HEP-benzaldehyde (99.7 mg; 2.5 μmol, nitrogen quantification). The mixture was gently rotated until all HEP-benzaldehyde had dissolved. During the following 2 hours, a 1M solution of sodium cyanoborohydride in MilliQ water was added in portions (5×50 μl), to reach a final concentration of 48 mM. Excess of GSC was then removed by dialysis as follows: the total reaction volume (5250 μl) was transferred to a dialysis cassette (Slide-A-Lyzer Dialysis Cassette, Thermo Scientific Prod#66810 with cut-off 10 kDa capacity: 3-12 ml). Solution was dialysed for 2 hours against 2000 ml of 25 mM Hepes buffer (pH 7.2) and once more for 17 h against 2000 ml of 25 mM Hepes buffer (pH 7.2). Complete removal of excess GSC from inner chamber was verified by HPLC on Waters X-Bridge phenyl column (4.6 mm×250 mm, 5 μm) and a water acetonitrile system (linear gradient from 0-85% acetonitrile over 30 min containing 0.1% phosphoric acid) using GSC as reference. Inner chamber material was collected and freeze dried to give 83% (nitrogen quantification) 41.5 kDa HEP-GSC as white powder. The HEP-GSC reagent made by this procedure contains a HEP polymer attached to sialic acid cytidine monophosphate via a 4-methylbenzoyl linkage.

### Example 17

### Synthesis of 21 kDa HEP-GSC Reagent with 4-Methylbenzoyl Sublinkage

This molecule was prepared using 21 kDa HEP-benzaldehyde and Glycyl sialic acid cytidine monophosphate (GSC) in a similar way as described for 41.5 kDa HEP-GSC above. Yield was 78% after freeze drying.

### Example 18

### Synthesis of 41.5 kDa HEP-[N]-FIX with 4-Methylbenzoyl Linkage

To FIX (12.3 mg) in 1 ml 10 mM histidine, 150 mM NaCl, 3 mM CaCl₂, pH 6.0 (reaction buffer) was added 16.7 μl of 1:2000 diluted in reaction buffer His-sialidase (AUS) (1.33 mg/ml, 83 U/mg before dilution), ST3GaIII (1.4 mg/ml, in 20 mM Hepes, 120 mM NaCl, 50% glycerol, pH 7.0) to a final concentration of 240 μg/ml reaction mixture, 99.3 μl of 41.5 kDa HEP-GSC (lyophilised compound reconstituted in the reaction buffer to a concentration of 100 mg/ml). pH of reaction mixture was adjusted to 6.0 with 18 μl 0.25 M HCl. The reaction mixture was incubated for 18 hours at 25° C. Unconjugated FIX, St3GaIIII and sialidase were separated from 41.5 kDa HEP-[N]-FIX in a flowthrough mode on a Source 30Q column equilibrated with 10 mM histidine buffer, 5 mM CaCl₂, 150 mM NaCl pH 6.0. 41.5 kDa H EP-[N]-FIX was eluted with a 0-100% gradient of elution buffer (10 mM Histidine, 5 mM CaCl₂, 1 M NaCl, pH 6.0) over 20 column volumes. N-acetylneuraminic acid cytidine monophosphate and St3GaIIII were added to the eluted pool to a final concentration of 0.8 mg/ml and 1 μg/ml, respectively and the reaction mixture was incubated at 25° C. for 3 hours. The 41.5 kDa HEP-[N]-FIX was then purified by affinity chromatography essentially as described in example 4 but with different buffers. The following buffers were used: Buffer A: 50 mM histidine, 100 mM NaCl, 10 mM CaCl_{2′} pH 6.2; buffer B: 50 mM histidine, 100 mM NaCl, 20 mM EDTA, pH 6.2. The isolated compound was dialyzed into 10 mM His, 150 mM NaCl, 5 mM CaCl₂, 0.005% Tween80, pH 6.4. 0.926 mg (7.5%) of 41.5 kDa HEP-[N]-FIX was isolated in 6.0 ml 10 mM His, 150 mM NaCl, 5 mM CaCl₂, 0.005% Tween80, pH 6.4 (0.15 mg/ml).

### Example 19

### Synthesis of 21 kDa HEP-[N]-FIX with 4-methylbenzoyl linkage

This compound was synthesised in similar way as described in example 18, using asialoFIX and 21 kDa HEP-GSC from example 16. The final conjugate contains a HEP polymer attached to FIX via a 4-methylbenzoyl linkage.

### Example 20

### Synthesis of Neuraminic Acid Cytidine Monophosphate Based 41.5 kDa HEP Conjugates with 4-Methylbenzoyl Linkage

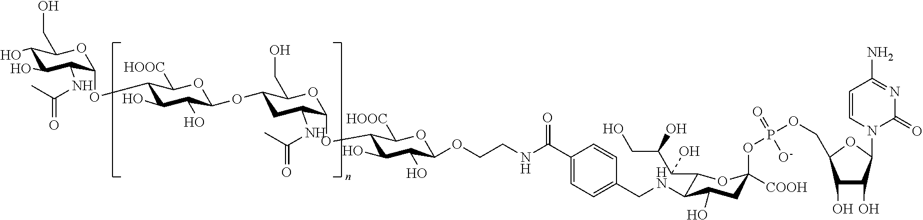

Neuraminic acid cytidine monophosphate is produced as described in Eur J Org Chem. 2000, 1467-1482. Reaction with HEP-aldehyde is performed as described in example 16, replacing GSC with neuraminic acid cytidine monophosphate. Neuraminic acid cytidine monophosphate (32 μmol) is dissolved in 50 mM Hepes, 100 mM NaCl, 10 mM CaCl₂ buffer, pH 7.0 buffer and added directly to dry 41.5 kDa HEP-benzaldehyde (2.5 μmol). The mixture is gently rotated until all HEP-benzaldehyde is dissolved. During the following 2 hours, a 1M solution of sodium cyanoborohydride in MilliQ water is added in portions to reach a final concentration of 48 mM. Excess of neuraminic acid cytidine monophosphate is then removed by dialysis as described in example 16. Complete removal of neuraminic acid cytidine monophosphate from inner chamber is verified by HPLC on Waters X-Bridge phenyl column (4.6 mm×250 mm, 5 μm) and a water acetonitrile system (linear gradient from 0-85% acetonitrile over 30 min containing 0.1% phosphoric acid) using neuraminic acid cytidine monophosphate as reference. Inner chamber material is then collected and freeze dried. The reagent made by this procedure contains a HEP polymer attached to sialic acid cytidine monophosphate via a 4-methylbenzoyl linkage.

### Example 21

### Synthesis of 9-amino-9-deoxy-N-acetylneuraminic acid cytidine monophosphate based HEP conjugates with 4-methylbenzoyl linkage

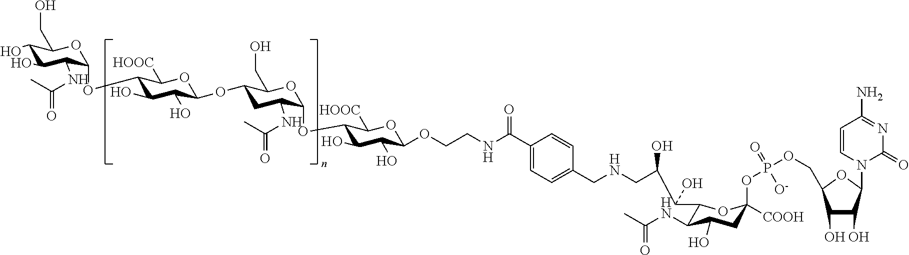

9-deoxy-amino N-acetylneuraminic acid cytidine monophosphate is produced as described in Eur J Biochem 168, 594-602 (1987). Reaction with HEP-aldehyde is performed as described in example 15, replacing GSC with 9-amino-9-deoxy-N-acetylneuraminic acid cytidine monophosphate. 9-Amino-9-deoxy-N-acetylneuraminic acid cytidine monophosphate (32 μmol) is dissolved in 50 mM Hepes, 100 mM NaCl, 10 mM CaCl₂ buffer, pH 7.0 buffer and added directly to dry 41.5 kDa HEP-benzaldehyde (2.5 μmol). The mixture is gently rotated until all HEP-benzaldehyde is dissolved. During the following 2 hours, a 1M solution of sodium cyanoborohydride in MilliQ water is added in portions to reach a final concentration of 48 mM. Excess of 9-amino-9-deoxy-N-acetylneuraminic acid cytidine monophosphate is then removed by dialysis as described in example 16. Complete removal of 9-amino-9-deoxy-N-acetylneuraminic acid cytidine monophosphate from inner chamber is verified by HPLC on Waters X-Bridge phenyl column (4.6 mm×250 mm, 5 μm) and a water acetonitrile system (linear gradient from 0-85% acetonitrile over 30 min containing 0.1% phosphoric acid) using 9-amino-9-deoxy-N-acetylneuraminic acid cytidine monophosphate as reference. Inner chamber material is collected and freeze dried. The reagent made by this procedure contains a HEP polymer attached to sialic acid cytidine monophosphate via a 4-methylbenzoyl linkage and is suitable for glycoconjugation with an asialo FIX glycoprotein.

### Example 22

### Synthesis of 2-keto-3-deoxy-nonic acid cytidine monophosphate based HEP conjugates with 4-methylbenzoyl linkage

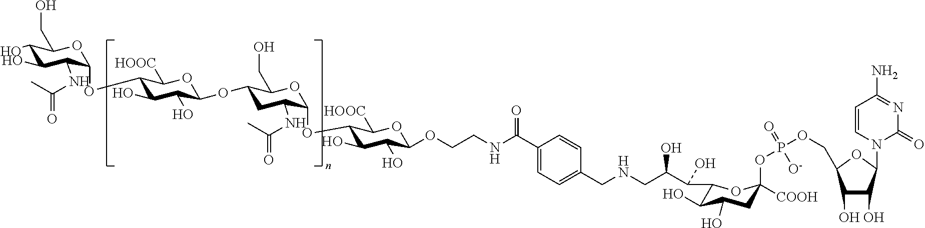

In a way similar to that shown in examples 20 and 21 HEP-sialic acid cytidine monophosphate reagent can be made starting from the sialic acid KDN. The initial amino derivatization at the 9-position is performed as described in Eur J Org Chem 2000, 1467-1482. Reaction with HEP-aldehyde is performed as described in example 16, replacing GSC with 9-amino-9-deoxy-2-keto-3-deoxy-nonic acid cytidine monophosphate. 9-amino-9-deoxy-2-keto-3-deoxy-nonic acid cytidine monophosphate (32 μmol) is dissolved in 50 mM Hepes, 100 mM NaCl, 10 mM CaCl₂ buffer, pH 7.0 buffer and added directly to dry 41.5 kDa HEP-benzaldehyde (2.5 μmol). The mixture is gently rotated until all HEP-benzaldehyde is dissolved. During the following 2 hours, a 1M solution of sodium cyanoborohydride in MilliQ water is added in portions to reach a final concentration of 48 mM. Excess of 9-amino-9-deoxy-2-keto-3-deoxy-nonic acid cytidine monophosphate is then removed by dialysis as described in example 15. Complete removal of 9-amino-9-deoxy-N-acetylneuraminic acid cytidine monophosphate from inner chamber is verified by HPLC on Waters X-Bridge phenyl column (4.6 mm×250 mm, 5 μm) and a water acetonitrile system (linear gradient from 0-85% acetonitrile over 30 min containing 0.1% phosphoric acid) using 9-amino-9-deoxy-2-keto-3-deoxy-nonic acid cytidine monophosphate as reference. Inner chamber material is collected and freeze dried. The reagent made by this procedure contains a HEP polymer attached to sialic acid cytidine monophosphate via a 4-methylbenzoyl linkage and is suitable for glycoconjugation with an asialoFIX glycoprotein.

### Example 23

### Pharmacokinetics of IV Dosed 60 kDa HEP-[C]-FIX(E162C) Compared to rFIX and 40 kDa PEG-[N]-FIX in FIX Deficient Mice

A pharmacokinetic study was performed in 45 FIX deficient mice F9 (Factor 9) knock-out (KO) mice (HB mice (B6.129P2-F9tm1Dws) originally obtained from D. W. Stafford (University of North Carolina)) after IV dosing with 27 nmol/kg equal to 1.5 mg FIX/kg of rFIX (BeneFIX®), 40 kDa PEG-[N]-FIX or of 60 kDa HEP-[C]-FIX(E162C). The dose was administered with 5 ml/kg in the tail vein and blood was collected from the orbital sinus by a capillary glass tube in a sparse sampling design resulting in 3 blood samples per mouse and three mice per time point at 0.08, 0.25, 0.5, 1, 4, 7, 17, 24, 30, 42, 48, 54, 72, 78, 96 hours after dosing. The blood was citrate stabilized and diluted 1:4 with a Hepes and BSA buffer of pH 7.4 and centrifuged for 5 minutes at 4000 RPM before the plasma was sent for analysis.

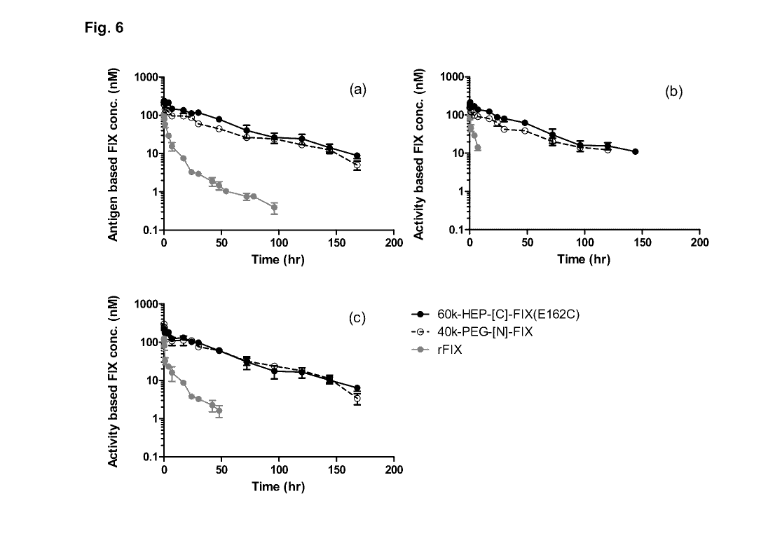

The plasma concentrations of FIX were determined with an antigen assay (LOCI), a chromogenic activity assay and by a clot assay, the results are shown in FIG. 6 and the pharmacokinetic parameters are shown in table 2.

The LOCI assay for hFIX was essentially build as the human insulin LOCI described by Poulsen, F & Jensen KB, J Biomol Screen 2007; 12(2):240-7. Briefly, the assay is a bead-based sandwich immunoassay with a broad analytical range for quantifying hFIX in human plasma. A 2-step reaction is performed incubating the sample with a mixture of biotinylated anti-FIX antibody and beads covalently coated with anti-FIX antibody. This was followed by incubation with beads covalently coated with streptavidin for 30 min. Light generated from a chemiluminescent reaction within the beads was quantitated. The antibodies used in the FIX LOCI assay were in-house produced Novo Nordisk monoclonal anti-FIX antibody and a polyclonal goat anti-hFIX antibody from LifeSpan BioSciences, Inc. (LS-B7226).

The commercial chromogenic assay kit was from Hypen Biophen (Hyphen Biomed (#221805)). Briefly, FIX from the sample was activated to FIXa by the addition of activated Factor XI (FXIa), Ca²⁺, phospholipids and activated Factor II (FIIa). FIXa was then complexed with supplied Factor VIII (FVIII) and phospholipids in the presence of Ca²⁺. The Tenase complex activates Factor X (FX) to Factor Xa (FXa). The formed FXa reacts with SXa-11 and pNA is released. pNA absorbs light at 405 nm. Except for FIX all reagents were added in surplus. Thus, the more FIX in the sample the more FXa is formed and the more pNA is released.

Coagulant activity in the plasma samples was estimated using a one-stage FIX clotting assay as described by Østergaard et al. Blood, 2011; 118:2333-41. The assay measures FIX activity-dependent time to fibrin clot formation. Briefly, equal amounts of test-sample, human FIX deficient plasma, APTT reagent (Synthafax), and CaCl₂ (0.02M) were used. Plasma samples were diluted 10 or 20 folds in a BSA containing HEPES buffer. The lower limit of quantification was approximately 70 U/L in plasma samples (10 fold diluted). Instrument and reagents were from Instrumentation Laboratories.

TABLE 2
Mean pharmacokinetic parameters of FIX variants after IV administration to F9-KO mice

| | | Cₘₐₓ | AUC_{0-∞} | V_{z} | CL | MRT | t½ |
|---|---|---|---|---|---|---|---|
| Assay | Compound | (nmol/L) | (hr * mol/L) | (L/kg) | (mL/hr/kg) | (hr) | (hr) |
| Antigen | 60 kDa HEP-[C]- | 237 | 10200 | 0.15 | 2.6 | 47.8 | 38.8 |
| | FIX(E162C) | | | | | | |
| Antigen | 40 kDa PEG-[N]-FIX | 219 | 6800 | 0.23 | 3.9 | 49.8 | 40.3 |
| Antigen | rFIX | 110 | 544 | 1.69 | 49.3 | 13.3 | 23.8 |
| Clot | 60 kDa HEP-[C]- | 217 | 7890 | 0.18 | 3.4 | 48.0 | 36.1 |
| | FIX(E162C) | | | | | | |
| Clot | 40 kDa PEG-[N]-FIX | 168 | 5180 | 0.28 | 5.2 | 51.2 | 38.1 |
| Clot | rFIX | 99 | 335 | 0.41 | 80.1 | 4.6 | 3.6 |
| Chromogen | 60 kDa HEP-[C]- | 222 | 8170 | 0.17 | 3.3 | 47.8 | 35.2 |
| | FIX(E162C) | | | | | | |
| Chromogen | 40 kDa PEG-[N]-FIX | 299 | 7770 | 0.17 | 3.5 | 49.8 | 33.5 |
| Chromogen | rFIX | 123 | 481 | 0.95 | 55.7 | 13.3 | 11.7 |

The parameters were calculated in a non-compartmental analysis based on sparse sampling, n = 3.
Cₘₐₓ: maximum concentration,
AUC_{0-∞}: area under the curve,
V_{z}: volume of distribution,
CL: clearance,
MRT: Mean Residence Time.

The mean plasma profiles as well as the pharmacokinetic parameters of 60 kDa HEP-[C]-FIX(E162C) and 40 kDa PEG-[N]-FIX were comparable in FIX-deficient mice (FIG. 6 and table 2. The half-life of 60 kDa HEP-[C]-FIX(E162C) was approximately 2 to 10 times longer than the half-life of rFIX, depending on the assay and the number of data points above lower limit of quantification (LLOQ) in the terminal elimination phase. The clearance of 60 kDa HEP-[C]-FIX(E162C) and 40 kDa PEG-[N]-FIX were approximately decreased by a factor 20 compared to the clearance of rFIX.

FIG. 6 shows plasma rFIX and FIX conjugate concentrations versus time in F9-KO mice. The concentrations were measured by an antigen based assay (a) as well as clot activity and chromogenic activity based assays (b) and (c), respectively) versus time. Results are mean±SD in a semi-logarithmic plot, n=3.

### Example 24

### Pharmacokinetics of IV Dosed 13 kDa-, 21 kDa-, 27 kDa- and 40 kDa HEP-FIX in FIX Deficient Mice

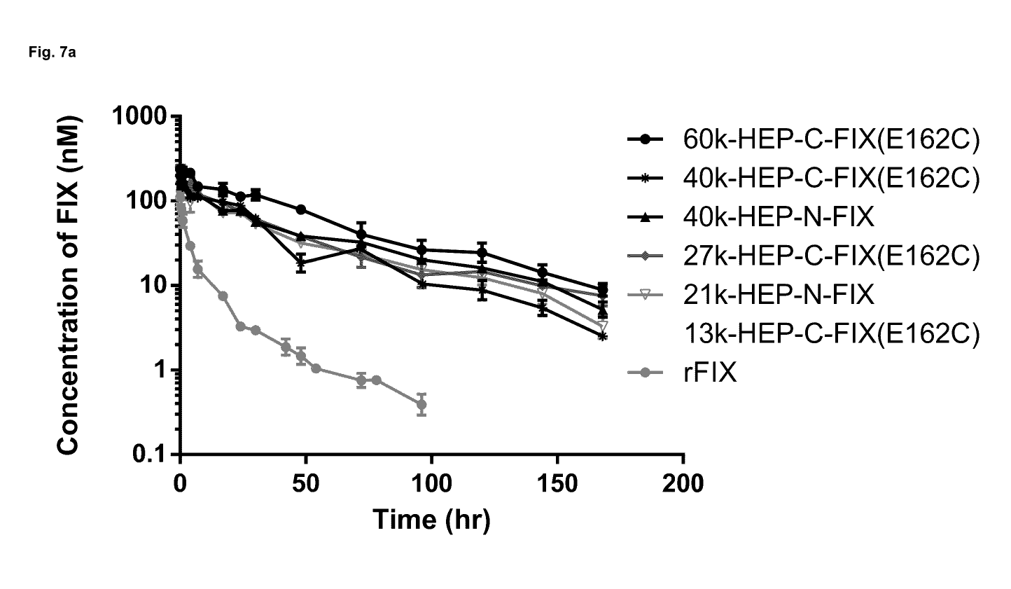

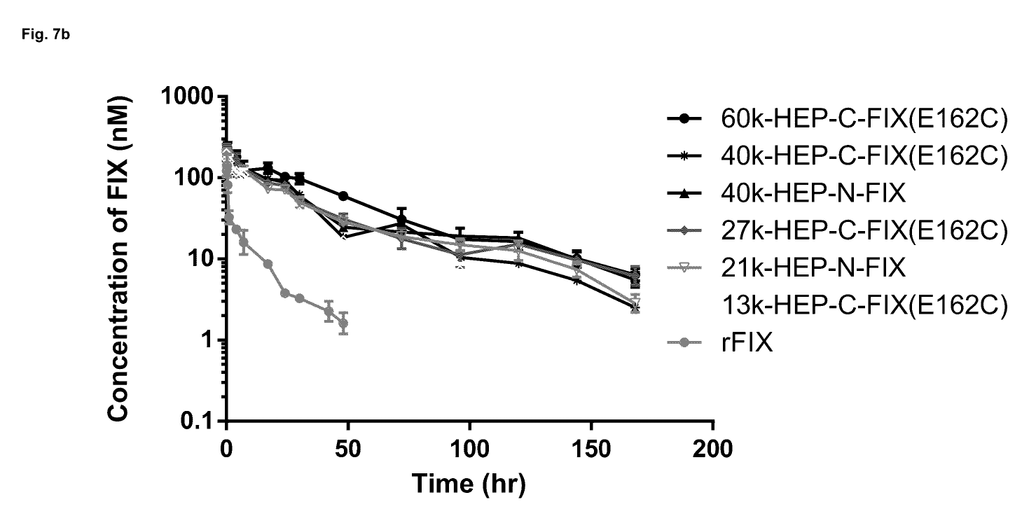

A pharmacokinetic study was performed in 75 FIX deficient mice (F9-KO mice) after IV dosing with 27 nmol/kg equal to 1.5 mg FIX/kg of 13 kDa HEP-[C]-FIX(E162C), 21 kDa HEP-[N]-FIX, 27 kDa HEP-[C]-FIX(E162C), 40 kDa HEP-[N]-FIX and 40 kDa HEP-[C]-FIX(E162C). The study was performed as described in example 23. The plasma was analysed with an antigen assay (LOCI) (a) and chromogenic activity assay (b), the results are shown in FIG. 7 and the pharmacokinetic parameters are shown in table 3 (also comprising the data on rFIX and 60 kDa HEP-[C]-FIX(E162C) (italic) from example 23 for comparison). Results are mean±SD in a semi-logarithmic plot, n=3.

The clearance of the FIX variants seemed to decrease with the size of the conjugated HEP polymer. Conjugation to HEP polymers of sizes between 13 and 60 kDa increased the half-life compared to rFIX to between 32 and 40 hours as measured in the antigen assay (cf. table 3).

TABLE 3
Mean pharmacokinetic parameters of FIX conjugated to 13, 20, 27, 40 and 60 kDa
HEP polymers after IV administration to F9-KO mice

| | | Cmax | AUC | Vz | CL | MRT | t½ |
|---|---|---|---|---|---|---|---|
| Assay | Compound | (nmol/L) | (hr * mol/L) | (L/kg) | (mL/hr/kg) | (hr) | (hr) |
| Antigen | 60 kDa HEP-[C]- | 237 | 10200 | 0.15 | 2.62 | 53.5 | 38.8 |
| | FIX(E162C) | | | | | | |
| Antigen | 40 kDa HEP-[C]- | 278 | 8540 | 0.159 | 3.14 | 48.9 | 35.0 |
| | FIX(E162C) | | | | | | |
| Antigen | 40 kDa HEP-[N]-FIX | 226 | 6590 | 0.238 | 4.07 | 56.0 | 40.6 |
| Antigen | 27 kDa HEP-[C]- | 210 | 6400 | 0.245 | 4.19 | 51.8 | 40.5 |
| | FIX(E162C) | | | | | | |
| Antigen | 21 kDa HEP-[N]-FIX | 236 | 5640 | 0.241 | 4.75 | 47.9 | 35.2 |
| Antigen | 13 kDa HEP-[C]- | 204 | 4070 | 0.303 | 6.58 | 38.7 | 31.9 |
| | FIX(E162C) | | | | | | |
| Antigen | rFIX | 110 | 544 | 1.69 | 49.3 | 13.3 | 23.8 |
| Chromogen | 60 kDa HEP-[C]- | 222 | 8170 | 0.17 | 3.28 | 47.8 | 35.2 |
| | FIX(E162C) | | | | | | |
| Chromogen | 40 kDa HEP-[C]- | 194 | 5350 | 0.221 | 5.01 | 41.1 | 30.5 |
| | FIX(E162C) | | | | | | |
| Chromogen | 40 kDa HEP-[N]-FIX | 262 | 6570 | 0.231 | 4.08 | 50.8 | 39.2 |
| Chromogen | 27 kDa HEP-[C]- | 207 | 5980 | 0.255 | 4.48 | 49.4 | 39.4 |
| | FIX(E162C) | | | | | | |
| Chromogen | 21 kDa HEP-[N]-FIX | 192 | 5350 | 0.249 | 5.01 | 46.5 | 34.5 |
| Chromogen | 13 kDa HEP-[C]- | 198 | 3760 | 0.300 | 7.13 | 35.5 | 29.2 |
| FIX(E162C) | | | | | | | |
| Chromogen | rFIX | 123 | 481 | 0.95 | 55.7 | 13.3 | 11.7 |

The parameters were calculated in a non-compartmental analysis based on sparse sampling, n = 3.
Cₘₐₓ: maximum concentration,
AUC_{0-∞}: area under the curve,
V_{z}: volume of distribution,
CL: clearance,
MRT: Mean Residence Time.

### Example 25

### Dose-Response of 60 kDa HEP-[C]-FIX(E162C) in F9-KO Mice

The effect of 60 kDa HEP-[C]-FIX(E162C) and FIX (Novo Nordisk A/S) was compared in a tail vein transection (TVT) model in F9-KO (Factor IX knock-out) mice (HB mice (B6.129P2-F9tm1Dws) originally obtained from D. W. Stafford (University of North Carolina). Briefly, F9-KO mice were dosed with increasing doses of 60 kDa HEP-[C]-FIX(E162C), rFIX or vehicle (5 ml/kg; 20 mM Hepes, 150 mM NaCl, 0.5% BSA pH 7.4), and after 10 minutes bleeding was induced by a template-guided transection of the left lateral tail vein at a tail diameter of 2.5 mm. The tail was immersed in temperate saline (37° C.) allowing visual recording of the bleeding for 60 min, where after the blood loss was determined by spectrophotometric measurement of the amount of lost haemoglobin Thus, erythrocytes were isolated by centrifugation at 4000×g for 5 min. The supernatant was discarded and the cells lysed with haemoglobin reagent (ABX Lysebio; ABX Diagnostics no. 906012, Triolab A/S, Broendby, Denmark). Cell debris was removed by centrifugation at 4000×g for 5 min. Samples were read at 550 nm and the total amount of haemoglobin was determined from a standard curve (HemoCue calibrator 707037, HemoCue, Vedbaek, Denmark).

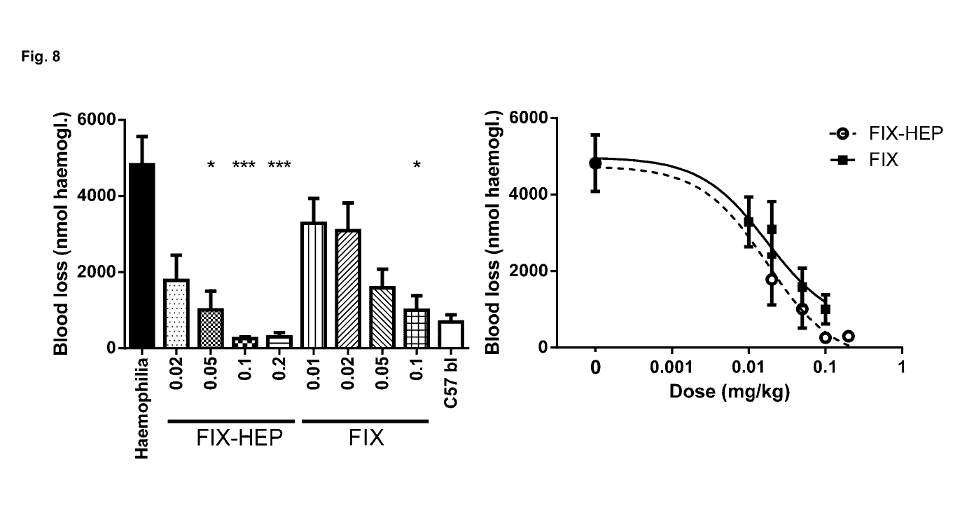

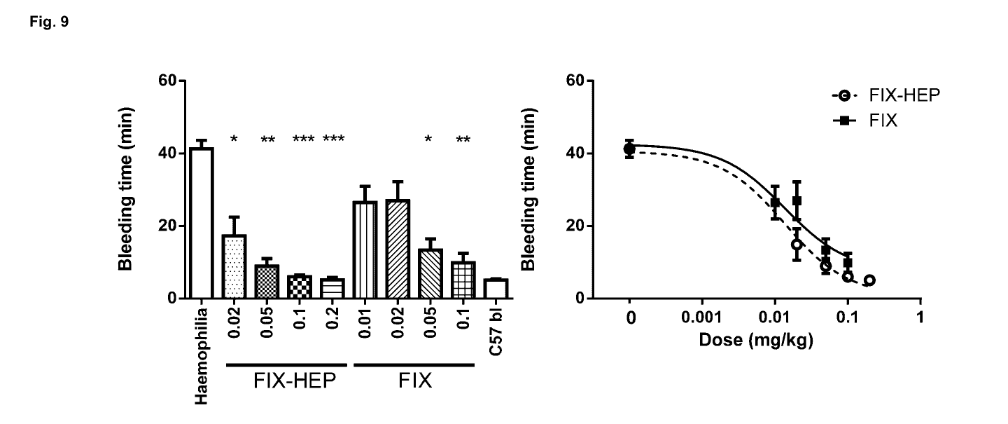

60 kDa HEP-[C]-FIX(E162C) significantly and dose-dependently reduced the blood loss, reaching normalization at 0.1 mg/kg (p<0.001; n=8). This was comparable with the effect of rFIX. Thus, the potency of 60 kDa HEP-[C]-FIX(E162C) and rFIX was comparable with an estimated ED₅₀ of 0.012 and 0.03 mg/kg 60 kDa HEP-[C]-FIX(E162C) and rFIX respectively (p=0.38; FIG. 8; table 4). FIG. 8 shows how 60 kDa HEP-[C]-FIX E162C (FIX-HEP) and rFIX dose-dependently and significantly reduced blood loss after tail vein transection in F9-KO mice with comparable potency. Similarly, the effect of 60 kDa HEP-[C]-FIX(E162C) and rFIX on bleeding time was comparable, with a significant and dose-dependent shortening in bleeding time with no significant difference in ED₅₀ for the two compounds (0.009 and 0.024 mg/kg, respectively; p=0.18; FIG. 9; table 4). FIG. 9 shows how 60 kDa HEP-[C]-FIX E162C (FIX-HEP) and rFIX dose-dependently and significantly reduced bleeding time after tail vein transection in F9-KO mice with comparable potency. The F9-KO mice were dosed 10 min before induction of bleeding. ED₅₀ was 0.009 mg/kg and 0.024 mg/kg for FIX-HEP and rFIX, respectively (p=0.18). *, ** and *** indicate statistical significant difference at p<0.05, 0.01 and 0.001, respectively, compared to the haemophilia control receiving vehicle. Data are mean±SEM. The F9-KO mice were dosed 10 min before induction of bleeding. ED₅₀ was 0.012 mg/kg and 0.030 mg/kg for FIX-HEP and rFIX, respectively (p=0.38). * and *** indicate statistical significant difference at p<0.05 and 0.001, respectively, compared to the haemophilia control group receiving vehicle. Data are mean±SEM.

TABLE 4
60 kDa HEP-[C]-FIX E162C (FIX-HEP) and rFIX dose-dependently reduced blood
loss and bleeding time in F9-KO mice.

| | | FIX-HEP (mg/kg) | | | | rFIX (mg/kg) | | | | C57/ |
|---|---|---|---|---|---|---|---|---|---|---|
| | Haem | 0.02 | 0.05 | 0.1 | 0.2 | 0.01 | 0.02 | 0.05 | 0.1 | BL |
| N | 8 | 8 | 8 | 8 | 8 | 8 | 8 | 8 | 8 | 8 |
| Blood loss | 4824 | 1780 | 1007* | 257*** | 300*** | 3286 | 3094 | 1587 | 1001* | 693 |
| (nmol/ | | | | | | | | | | |
| haemogl.) | | | | | | | | | | |
| SEM | 743 | 668 | 494 | 45 | 107 | 652 | 724 | 488 | 380 | 184 |
| Bleeding | 41 | 17.3* | 9.0** | 6.0*** | 5.2*** | 26.5 | 27.0 | 13.4* | 9.9** | 5.1 |
| time (min) | | | | | | | | | | |
| SEM | 2.4 | 5.2 | 2.1 | 0.53 | 0.67 | 4.5 | 5.2 | 3.1 | 2.6 | 0.31 |

60 kDa HEP-[C]-FIX E162C (HEP-FIX) and rFIX dose-dependently and significantly reduced blood loss and bleeding time after tail vein transection in F9-KO mice. The F9-KO mice were dosed 10 min before induction of bleeding. *, ** and ***indicate statistical significant difference at p < 0.05, 0.01 and 0.001, respectively, compared to the haemophilia control group receiving vehicle. ‘Haem’ refers to F9-KO mice treated with control vehicle. C57/BL refers to wild type mice treated with control vehicle.

### Example 26

### Dose-Response of 40 kDa HEP-[N]-FIX in F9-KO Mice

The effect of 40 kDa HEP-[N]-FIX and rFIX (Novo Nordisk A/S) was compared in a tail vein transection (TVT) model in F9-KO (Factor IX knock-out) mice (Haemophilia B mice (B6.129P2-F9tm1Dws)) originally obtained from D. W. Stafford (University of North Carolina). Briefly, F9-KO mice were dosed with increasing doses of 40 kDa HEP-[N]-FIX, rFIX or vehicle (5 ml/kg; 10 mM Histidine, 150 mM NaCl, 5 mM CaCl₂, 0.005% Tween80, pH 6.4), and after 10 minutes bleeding was induced by a template-guided transection of the left lateral tail vein at a tail diameter of 2.5 mm. The tail was immersed in temperate saline (37° C.) allowing visual recording of the bleeding for 60 min, where after the blood loss was determined by spectrophotometric measurement of the amount of lost haemoglobin Thus, erythrocytes were isolated by centrifugation at 4000×g for 5 min. The supernatant was discarded and the cells lysed with haemoglobin reagent (ABX Lysebio; ABX Diagnostics no. 906012, Triolab A/S, Broendby, Denmark). Cell debris was removed by centrifugation at 4000×g for 5 min. Samples were read at 550 nm and the total amount of haemoglobin was determined from a standard curve (HemoCue calibrator 707037, HemoCue, Vedbaek, Denmark).

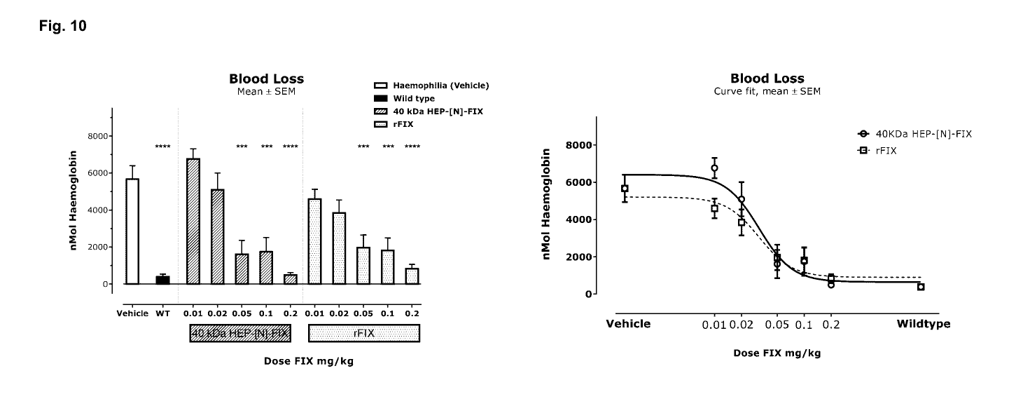

Both 40 kDa HEP-[N]-FIX and rFIX significantly and dose-dependently reduced the blood loss, reaching full response at 0.2 mg/kg. By two-way ANOVA analysis, no significant difference between the effect of the compounds was observed (P=0.1924), but a significant (P<0.0001) effect of dose was detected. Thus, the potency of 40 kDa HEP-[N]-FIX and rFIX was comparable, with no significant difference between estimated ED₅₀ values of 0.032 mg/kg and 0.027 mg/kg, respectively (p=0.69; FIG. 10, table 5).

FIG. 10 shows how 40 kDa HEP-[N]-FIX and rFIX dose-dependently and significantly reduced the blood loss after tail vein transection in F9-KO mice with comparable potency.

The F9-KO mice were dosed 10 min before induction of bleeding. ED₅₀ was 0.032 mg/kg and 0.027 mg/kg for 40 kDa HEP-[N]-FIX and rFIX, respectively (p=0.67). *** and **** indicate statistical significant difference at p<0.001 and 0.0001, respectively, compared to the haemophilia control receiving vehicle. Data are mean±SEM.

Similarly, the effect of 40 kDa HEP-[N]-FIX and rFIX on bleeding time was comparable: by two-way ANOVA, no significant difference between the effect of the compounds was observed (P=0.82), but a significant (P<0.0001) effect of dose was detected. Thus, a significant and dose-dependent shortening in bleeding time was observed for 40 kDa HEP-[N]-FIX and rFIX, with no significant difference in estimated ED₅₀ (0.028 and 0.034 mg/kg, respectively; p=0.57; table 5).

TABLE 5
40 kDa HEP-[N]-FIX and rFIX dose-dependently reduced blood loss and bleeding time in F9-KO mice

| | | N40 kDa HEP-[N]-FIX (mg/kg) | | | | | rFIX (mg/kg) | | | | | |
|---|---|---|---|---|---|---|---|---|---|---|---|---|
| | Haem | 0.01 | 0.02 | 0.05 | 0.1 | 0.2 | 0.01 | 0.02 | 0.05 | 0.1 | 0.2 | Wild type |
| N | 8 | 8 | 8 | 8 | 8 | 8 | 8 | 8 | 8 | 8 | 8 | 6 |
| Blood | 5670 | 6763 | 5088 | 1607*** | 1745*** | 491**** | 4592 | 3844 | 1967*** | 1812*** | 823**** | 385**** |
| loss | | | | | | | | | | | | |
| (nmol/ | | | | | | | | | | | | |
| Haem- | | | | | | | | | | | | |
| ogl.) | | | | | | | | | | | | |
| SEM | 728 | 549 | 915 | 494 | 766 | 130 | 528 | 697 | 684 | 679 | 241 | 147 |
| Bleed- | 33.1 | 37.0 | 23.5 | 8.48**** | 8.25*** | 5.07**** | 27.3 | 27.8 | 13.5** | 9.9*** | 6.6**** | 2.90**** |
| ing | | | | | | | | | | | | |
| time | | | | | | | | | | | | |
| (min) | | | | | | | | | | | | |
| SEM | 4.0 | 2.8 | 5.7 | 3.64 | 2.56 | 0.31 | 3.9 | 4.7 | 2.3 | 2.6 | 1.7 | 0.39 |

**, *** and ****indicate statistical significant difference at p < 0.01, 0.001 and 0.001, respectively, compared to the haemophilia control group receiving vehicle. ‘Haem’ refers to F9-KO mice treated with control vehicle.

### Example 27

### Performance of HEP-Factor IX Conjugates in One-Stage Clotting Assays

PEGylation of proteins can affect the clotting times in one-stage clotting assays depending on the aPTT reagent used (Leong et al. J Thromb Haemost 2011; 9 (Suppl 2):379 (P-TU-223)) and for N9-GP (nonacog beta pegol; glycoPEGylated recombinant FIX) PEGylation can result in large variability in such assays.

The performance of HEP-FIX conjugates relative to BeneFIX® and N9-GP was evaluated in five different one-stage clot assays and in a two-stage chromogenic assay as follows. Three concentrations (5, 15 and 45 nM, respectively) of the following FIX compounds were spiked into human FIX depleted plasma (Affinity Biologicals): 27 kDa HEP-[C]-FIX (E162C), 40 kDa HEP-[N]-FIX, 40 kDa HEP-[C]-FIX (E162C), 60 kDa HEP-[C]-FIX (E162C), N9-GP and BeneFIX®. FIX activity of the spiked samples was measured using a two-stage chromogenic assay, Biophen Factor IX, according to the manufacture's instruction (Hyphen Biomed). This result was defined as 100% activity, because the chromogenic assay is neutral towards the polymer attachment.

Clotting activity was measured in the same samples in one-stage clotting assays using the following aPTT reagents; Dade Actin® FS (Siemens), STA PTT® (Stago), APTT SP (ILS), Synthafax® (ILS), Synthasil® (ILS). Briefly, equal amounts of test-sample, human FIX deficient plasma, APTT reagent, and CaCl₂ (0.02M) were used. The assay measures FIX activity-dependent time to fibrin clot formation measured on a coagulation analyser from ILS. A pool of normal human plasma (ILS) that had been calibrated against the international plasma standard (NIBSC) was used as calibrator. The measured activity was compared to the activity measured in the chromogenic assay and results were given in percentage of chromogenic activity.

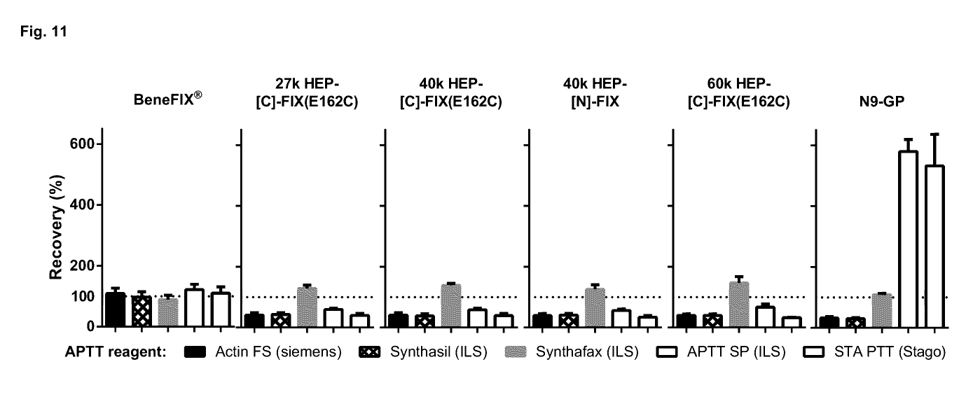

Results: The performance of HEP-FIX conjugates in the one-stage clotting assays was evaluated by calculating the recovery of FIX activity in the spiked human samples, as defined by the chromogenic method. Results are listed in table 6 and illustrated in FIG. 11. The performance of BeneFIX® was as expected; some variation was observed but recovery of FIX activity with all five aPTT reagents was between 89-122% (i.e. 33 percentage points). The recovery of N9-GP activity on the other hand showed a large variability and recovery ranged from 30% to 553% (i.e. 523 percent points) with the five aPTT reagents used. The recovery of the HEP-FIX conjugate activity was in the 32-147% range (i.e. 115 percentage points) and the assay performance of the HEP-FIX conjugates was thus improved compared with the assay performance of N9-GP for these five aPTT reagents. The assay performance was not majorly affected by length of the HEP polymer, nor by the polymer attachment point on FIX. Table 6 and FIG. 11 show recovery of FIX activity in spiked human FIX deficient plasma relative to chromogenic activity. Three concentrations of compounds (5, 15 and 45 nM, respectively) were spiked into human FIX depleted plasma and analysed using the Biophen Hypen chromogenic assay and five specified aPTT reagents in the one-stage clot assay. Results are given as clot activity in percent of chromogenic activity and are mean+/−SD, n=3. Activity was measured against a normal human plasma calibrator (ILS) in all assays.

TABLE 6
Recovery of FIX activity in spiked human FIX deficient
plasma relative to chromogenic activity

| | Synthafax ® | Actin FS ® | Synthasil ® | APTT SP | STA PIT ® |
|---|---|---|---|---|---|
| | (ILS) | (Siemens) | (ILS) | (ILS) | (Stago) |
| BeneFIX ® | 89 ± 15 | 109 ± 18 | 97 ± 18 | 122 ± 17 | 110 ± 21 |
| 27 kDa HEP-[C]- | 129 ± 11 | 41 ± 8 | 43 ± 6 | 59 ± 5 | 40 ± 7 |
| FIX(E162C) | | | | | |
| 40 kDa HEP-[C]- | 139 ± 6 | 41 ± 8 | 38 ± 7 | 58 ± 6 | 39 ± 7 |
| FIX(E162C) | | | | | |
| 40 kDa HEP-[N]-FIX | 126 ± 15 | 39 ± 7 | 41 ± 5 | 56 ± 5 | 34 ± 6 |
| 60 kDa HEP-[C]- | 147 ± 20 | 40 ± 5 | 40 ± 5 | 67 ± 10 | 32 ± 2 |
| FIX(E162C) | | | | | |
| N9-GP | 109 ± 4 | 32 ± 5 | 30 ± 4 | 580 ± 40 | 553 ± 103 |

### Example 28

### Duration of Effect of 40 kDa HEP-[N]-FIX in F9-KO Mice

The duration of effect of 40 kDa HEP-[N]-FIX and rFIX (Novo Nordisk A/S) was compared in a tail vein transection (TVT) model in F9-KO (Factor IX knock-out) mice (Haemophilia B mice (B6.129P2-F9tm1Dws)) originally obtained from D. W. Stafford (University of North Carolina). Briefly, F9-KO mice were dosed with 0.4 mg/kg (approx. 80 IU/kg) of 40 kDa HEP-[N]-FIX, an equivalent dose of rFIX or vehicle (5 ml/kg; 10 mM Histidine, 150 mM NaCl, 5 mM CaCl₂, 0.005% Tween80, pH 6.4). Bleeding was induced by a template-guided transection of the left lateral tail vein at a tail diameter of 2.5 mm at either 0, 48, 72, 120 or 168 hours after dosing. The tail was immersed in temperate saline (37° C.) allowing visual recording of the bleeding for 60 min, where after the blood loss was determined by spectrophotometric measurement of the amount of lost haemoglobin Thus, erythrocytes were isolated by centrifugation at 4000×g for 5 min. The supernatant was discarded and the cells lysed with haemoglobin reagent (ABX Lysebio; ABX Diagnostics no. 906012, Triolab A/S, Broendby, Denmark). Cell debris was removed by centrifugation at 4000×g for 5 min. Samples were read at 550 nm and the total amount of haemoglobin was determined from a standard curve (HemoCue calibrator 707037, HemoCue, Vedbaek, Denmark).

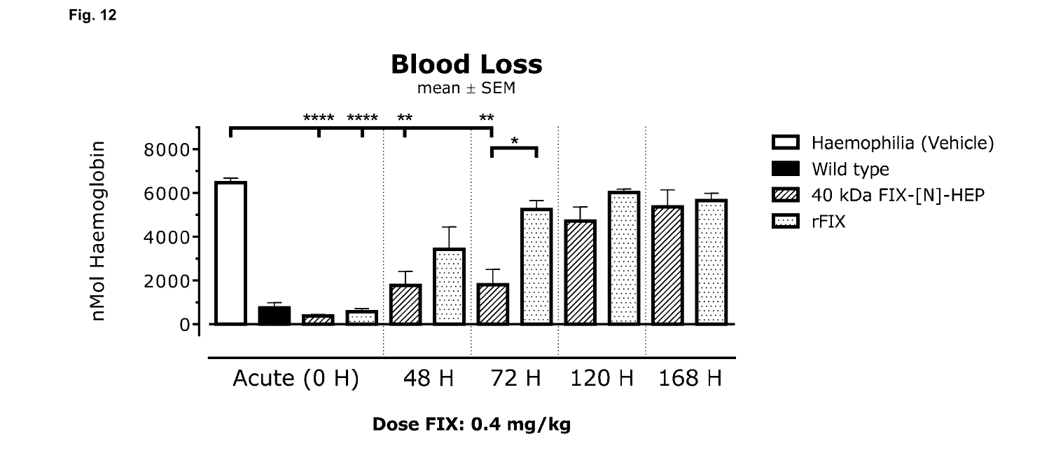

A time dependent effect on blood loss was observed (FIG. 12); statistical analysis was performed by one-way ANOVA with Bonferroni's correction for multiple comparisons (table 7). Compared to the vehicle group, both 40 kDa HEP-[N]-FIX and rFIX significantly (P<0.0001) reduced the blood loss in the acute setting, 0 hours after dosing. Animals treated with 40 k-HEP-[N]-FIX also bled significantly less than vehicle animals 48 hours (P=0.0012) and 72 hours (P=0.0028) after dosing, whereas animals treated with rFIX did not. 72 hours after injection, the bleeding response significantly differed between the compounds (P=0.020). At 168 hours after dosing, the effect of both compounds was no longer detectable.

Similarly, the effect of 40 kDa HEP-[N]-FIX and rFIX on bleeding time was observed (table 7). Compared to the vehicle group, both 40 kDa HEP-[N]-FIX and rFIX significantly (P<0.0001) reduced bleeding time 0 hours after dosing, and 40 kDa-HEP-[N]-FIX reduced bleeding time 48 hours (P=0.0060) and 72 hours (P=0.0041) after dosing, whereas rFIX did not. 72 hours after injection, bleeding time significantly differed between the compounds (P=0.022). At 168 hours after dosing, the effect of both compounds was no longer detectable.

Thus, 40 kDa-HEP-[N]-FIX and rFIX exhibited comparable haemostatic effects immediately after dosing, however the effects persisted significantly longer for 40 kDa-HEP-[N]-FIX than for a comparable dose of rFIX.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

TABLE 7
A 0.4 mg/kg dose of 40 kDa HEP-[N]-FIX had significantly longer duration of effect on blood loss and bleeding time than a
comparable dose of rFIX

| | | 40 kDa HEP-[N]-FIX (hours after dosing) | | | | | rFIX (hours after dosing) | | | | | |
|---|---|---|---|---|---|---|---|---|---|---|---|---|
| | Haem | 0 | 48 | 72 | 120 | 168 | 0 | 48 | 72 | 120 | 168 | Wild type |
| N | 16 | 8 | 8 | 8 | 8 | 8 | 8 | 8 | 8 | 8 | 8 | 6 |
| Blood loss | 6472 | 374.3**** | 1776** | 1807** | 4717 | 5363 | 570.5**** | 3241 | 5248 | 6018 | 5657 | 750.9 |
| (nmol/ | | | | | | | | | | | | NT |
| Haemogl.) | | | | | | | | | | | | |
| SEM | 207 | 69.0 | 635 | 701 | 644 | 783 | 140 | 1022 | 406 | 158 | 326 | 236 |
| Bleeding | 39.1 | 5.1**** | 10.0** | 15.0** | 28.2 | 30.7 | 5.1**** | 24.3 | 35.2 | 39.8 | 38.2 | 2.90 |
| time (min) | | | | | | | | | | | | NT |
| SEM | 1.7 | 0.22 | 6 2.3 | 4.0 | 4.5 | 4.7 | 0.60 | 5.9 | 3.1 | 2.9 | 4.5 | 0.29 |

Equivalent doses (0.4 mg/kg or approx. 80 IU/kg) of 40 kDa HEP-[N]-FIX or rFIX reduced blood loss and bleeding time after tail vein transection in F9-KO mice in a time dependent manner.
**, *** and ****indicate statistical significant difference at p < 0.01, 0.001 and 0.0001, respectively, compared to the haemophilia control group receiving vehicle. ‘Haem’ refers to F9-KO mice treated with control vehicle.
NT = Not tested.

1. A conjugate comprising a Factor IX polypeptide, a linking moiety, and a heparosan polymer;
wherein the linking moiety comprises X and connects the Factor IX polypeptide and the heparosan polymer as follows:
[heparosan polymer]-[X]-[Factor IX polypeptide];
wherein X comprises a sialic acid derivative which connects a moiety according to Formula 1 below to the Factor IX polypeptide: 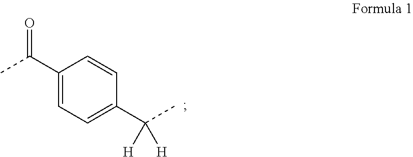
and
wherein the sialic acid derivative is a glycyl sialic acid according to Formula 2 below: 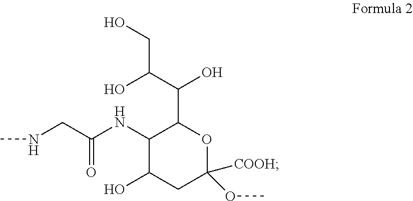
and
wherein the moiety of Formula 1 is connected to the terminal —NH handle of the glycyl sialic acid of Formula 2.

2. (canceled)

3. The conjugate according to claim 1, wherein the
[heparosan polymer]-[X]—
comprises the structural fragment shown in Formula 3 below: 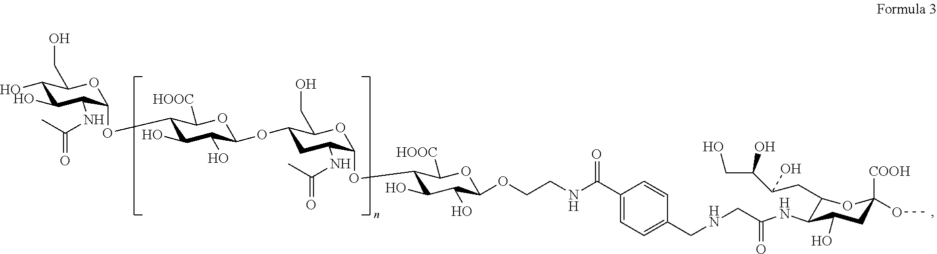
and
wherein n is an integer from 5 to 450.

4. A conjugate comprising a Factor IX polypeptide and a heparosan polymer, wherein said heparosan polymer has a molecular weight in the range 5 to 100 kDa.

5. The conjugate according to claim 4, wherein the heparosan polymer has a molecular weight in the range 13 to 60 kDa.

6. The conjugate according to claim 4, wherein the heparosan polymer has a molecular weight in the range 27 to 40 kDa.

7. The conjugate according to claim 4, wherein the molecular weight of the heparosan polymer is 40 kDa+/−10%.

8. A pharmaceutical composition comprising the conjugate according to any of claims 1 to 7.

9. A method of performing a aPTT assay, wherein the inter-assay variability in recovery of Factor IX activity is less than 523 percentage points.

10. The method according to claim 9, wherein the inter-assay variability in recovery of Factor IX activity is no more than 115 percentage points.

11. (canceled)

12. A method of treating haemophilia B, comprising administering the conjugate according to any one of claims 1, 3, 4, 5, 6, and 7 to a patient in need thereof.

13. The method according to claim 12, wherein the treating is prophylactic treatment.

14. A method of conjugating a heparosan polymer to a Factor IX polypeptide, comprising the steps of:
a) reacting a heparosan polymer comprising a reactive amine [HEP-NH] with an activated 4-formylbenzoic acid to yield the compound of Formula 4 below, 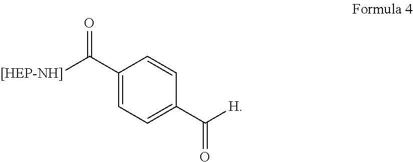
wherein the [HEP-NH] is a HEP polymer functionalized with a terminal primary amine;
b) reacting the compound of Formula 4 with a CMP-activated sialic acid derivative under reducing conditions; and
c) conjugating the compound obtained in step b) to a glycan on the Factor IX polypeptide.

15. (canceled)
